(19) United States
(12) Patent Application Publication
(10) Pub. No.: US 2025/0229067 A1
(43) Pub. Date: Jul. 17, 2025

(54) FILTER WITH A MICROFLUIDIC PUMP OR VALVE

(71) Applicant: Boston Scientific Scimed, Inc., Maple Grove, MN (US)

(72) Inventors: Thomas Sinnott, Enniscorthy (IE); Daragh Nolan, Youghal (IE); Richard Percy, Leamlara (IE); Matthew Lee Nelson, Plymouth, MN (US)

(21) Appl. No.: 19/012,156

(22) Filed: Jan. 7, 2025

Related U.S. Application Data
(60) Provisional application No. 63/620,267, filed on Jan. 12, 2024.

(51) Int. Cl. A61M 29/02

(52) U.S. Cl. CPC A61M 29/02; A61M 2205/0216; A61M 2205/0238; A61M 2205/04; A61M 2205/7545

(57) ABSTRACT

An implantable fluid-operated device controls fluid flow between a fluid reservoir and an inflatable member. The device includes a base plate; a deformable diaphragm; a fluid chamber between the base plate and the deformable diaphragm; a fluid filter; and a piezoelectric element. The base plate defines fluid passageways for routing fluid between the fluid chamber and the fluid reservoir and the inflatable member. The fluid filter is disposed within or at an end of a fluid passageway. The piezoelectric element is attached to the deformable diaphragm, and the piezoelectric element is operable to repeatedly change a volume of the fluid chamber by deforming the deformable diaphragm to pump fluid from the fluid reservoir to the inflatable member.

### CROSS-REFERENCE TO RELATED APPLICATION

This application claims priority to U.S. Provisional Patent Application No. 63/620,267, filed on Jan. 12, 2024, entitled “FILTER WITH A MICROFLUIDIC PUMP OR VALVE”, the disclosure of which is incorporated by reference herein in its entirety.

### TECHNICAL FIELD

This disclosure relates generally to bodily implants, and more specifically to bodily implants including a fluid control system having one or more pumps and/or valves that include a fluid filter.

### BACKGROUND

Active implantable fluid-operated inflatable devices can include one or more pumps that regulate a flow of fluid between different portions of the implantable device. One or more valves can be positioned within fluid passageways of the device to direct and control the flow of fluid to achieve inflation, deflation, pressurization, depressurization, activation, deactivation and the like of different fluid-filled components of the device. In some implantable fluid-operated devices, an implantable pumping device may be manually operated by the user to provide for the transfer of fluid between a reservoir and the fluid-filled implant components of the device. In some situations, manual operation of the pumping device may make it difficult to achieve consistent inflation, deflation, pressurization, depressurization, activation, deactivation and the like of the fluid-filled implant components. Inconsistent inflation, deflation, pressurization, depressurization, activation and/or deactivation of the fluid-filled implant device(s) may adversely affect patient comfort, efficacy of the device, and the overall patient experience. Some implantable fluid-operated devices include an electronic control system including an electronically controlled manifold providing for the transfer of fluid within the implantable fluid-operated device. The use of the electronic control system may provide for more accurate actuation and control of the flow of fluid between components of the inflatable device, thus improving performance and efficacy of the device, as well as patient comfort and safety. The electronic control system may include one or more electronically operated pumps and one or move valves to control the flow of fluid in the system, and the pumps and valve may have relatively narrow fluid passageway. Foreign (e.g., particulate) matter introduced into the system may contaminate the fluid that is used in the system, which may lead to poor performance or malfunctioning of the pumps and or valves. Thus, a need exists to control the introduction of foreign matter into the pumps and valves and to mitigate the effects of any foreign matter introduced into the system on the performance and longevity of the pumps and valves.

### SUMMARY

In a general aspect, the techniques described herein relate to an implantable fluid-operated device configured to control fluid flow between a fluid reservoir and an inflatable member. The device includes a base plate; a deformable diaphragm; a fluid chamber; a fluid filter; and a piezoelectric element. The fluid chamber is defined between the base plate and the deformable diaphragm, and the fluid chamber is in fluidic connection with the fluid reservoir and with the inflatable member. The base plate defines a first fluid passageway configured for routing fluid from the fluid reservoir into the fluid chamber and a second fluid passageway configured for routing fluid from the fluid chamber to the inflatable member. The fluid filter is disposed within or at an end of the first fluid passageway or disposed within or at an end of the second fluid passageway, where the end of the first fluid passageway is distal from the fluid chamber and where the end of the second fluid passageway is distal from the fluid chamber. The piezoelectric element is attached to the deformable diaphragm, and the piezoelectric element is operable to repeatedly change a volume of the fluid chamber by deforming the deformable diaphragm to pump fluid from the fluid reservoir to the inflatable member.

Implementations can include one or more of the following features, alone or in any combination with each other.

For example, the fluid filter can include a metal foil having a pattern of openings configured to permit fluid to flow through the openings and configured to block particulates having a characteristic size larger than a threshold size from flowing through the openings.

In another example, the metal foil can include titanium.

In another example, the base plate can include titanium and where the metal foil is welded to the base plate with openings of the pattern of openings being located at the end of the first fluid passageway or at the end of the second fluid passageway.

In another example, the pattern of openings can be stamped into the metal foil.

In another example, the pattern of openings can be laser-etched into the metal foil.

In another example, the pattern of openings can be chemically etched into the metal foil.

In another example, the filter can be a gyroid filter.

In another example, an average path distance of fluid through the gyroid filter can be more than two times a thickness of the gyroid filter in a direction parallel to an average flow direction of fluid through the gyroid filter.

In another example, the gyroid filter can include ceramic material.

In another example, the gyroid filter can be located within the first fluid passageway.

In another example, the gyroid filter can be located within the second fluid passageway.

In another example, inner walls of the gyroid filter can include a hydrophobic coating.

In another example, first fluid passageway can be tapered, such that it has a larger cross-sectional area at the end of the first fluid passageway that is distal to the fluid chamber than an end of the first fluid passageway that is proximate to the fluid chamber.

In some aspects, the techniques described herein relate to an implantable fluid-operated device configured to control fluid flow between a fluid reservoir and an inflatable member. The device includes: a titanium base plate, where the base plate defines a first fluid passageway through the base plate and defines a second fluid passageway through the base plate; a diaphragm coupled to the base plate; a fluid chamber defined between the base plate and the diaphragm; a first opening formed in the base plate, the first opening connecting the fluid chamber to a first fluid passageway; a second opening formed in the base plate, the second opening connecting the fluid chamber to a second fluid passageway; a fluid control device provided at one of the first opening or the second opening; and a fluid filter. In an open state of the fluid control device, the first fluid passageway, the fluid chamber, and the second fluid passageway are fluidically connected to guide fluid through the implantable fluid-operated device in a first fluid flow direction, and, in a closed state of the fluid control device, the fluid control device blocks the flow of fluid the flow of fluid between the first fluid passageway and the second fluid passageway in response to a force applied in a second fluid flow direction, opposite the first fluid flow direction. The fluid filter is disposed within or at an end of the first fluid passageway or disposed within or at an end of the second fluid passageway, where the end of the first fluid passageway is distal from the fluid chamber and where the end of the second fluid passageway is distal from the fluid chamber.

Implementations can include one or more of the following features, alone or in any combination with each other.

For example, the fluid filter can include a titanium foil having a pattern of openings configured to permit fluid to flow through the openings and configured to block particulates having a characteristic size larger than a threshold size from flowing through the openings, where titanium foil is welded to the base plate with openings of the pattern of openings being located at the end of the first fluid passageway or at the end of the second fluid passageway.

In another example, the filter can be a gyroid filter.

In another example, an average path distance of fluid through the gyroid filter can be more than two times a thickness of the gyroid filter in a direction parallel to an average flow direction of fluid through the gyroid filter.

In another example, inner walls of the gyroid filter can include a hydrophobic coating.

In another example, first fluid passageway can be tapered, such that it has a larger cross-sectional area at the end of the first fluid passageway that is distal to the fluid chamber than an end of the first fluid passageway that is proximate to the fluid chamber.

### BRIEF DESCRIPTION OF THE DRAWINGS

FIG. 1 is a block diagram of an implantable fluid-operated inflatable device.

FIG. 2 illustrates a system including an example implantable fluid-operated inflatable device.

FIG. 3A is a schematic diagram of a fluidic architecture of an implantable fluid-operated inflatable device.

FIG. 3B is a schematic diagram of a fluidic architecture of an implantable fluid-operated inflatable device.

FIG. 4A is an exploded view of an example valve device of a fluid control system of a fluid-operated inflatable device.

FIG. 4B is another exploded view of the example valve device shown in FIG. 4A.

FIG. 4C is a cross-sectional view of the example valve device shown in FIG. 4A, in a closed position.

FIG. 4D is a cross-sectional view of the example valve device shown in FIG. 4A, in an open position.

FIG. 4E is a top view of the example valve device shown in FIG. 4A.

FIG. 4F is a microscopic image of a surface of the diaphragm having an erratic texture pattern on the surface.

FIG. 4G is a microscopic image of a surface of the diaphragm having a periodic texture pattern on the surface.

FIG. 4H is a flowchart of a process for assembling components of a valve device.

FIG. 5A is a schematic view of an example valve device including an example auxiliary flow control device, with the example valve device in an open position.

FIG. 5B is a schematic view of an example valve device including an example auxiliary flow control device, with the example valve device in a closed position.

FIG. 6A illustrates an example auxiliary flow control device, in an open position.

FIG. 6B illustrates the example auxiliary flow control device shown in FIG. 6A, in a closed position.

FIG. 6C is an exploded perspective view of the example auxiliary flow control device shown in FIGS. 6A and 6B relative to a base plate of an example valve device.

FIG. 6D is an exploded perspective view of an example auxiliary flow control device relative to a base plate of an example valve device.

FIG. 7A illustrates an example valve device including an example auxiliary flow control device, in an open position.

FIG. 7B illustrates the example valve device including the example auxiliary flow control device shown in FIG. 7A, in a closed position.

FIG. 7C is a perspective view of the example auxiliary flow control device shown in FIGS. 7A and 7B.

FIG. 8A illustrates an example valve device including an example auxiliary flow control device, in an open position.

FIG. 8B illustrates the example valve device including the example auxiliary flow control device shown in FIG. 8A, in a closed position.

FIG. 8C is a perspective view of the example auxiliary flow control device shown in FIGS. 8A and 8B.

FIG. 9A illustrates an example valve device including an example auxiliary flow control device, in an open position.

FIG. 9B illustrates the example valve device including the example auxiliary flow control device shown in FIG. 9A, in a closed position.

FIG. 9C is a perspective view of the example auxiliary flow control device shown in FIGS. 9A and 9B.

FIG. 10A is an exploded view of an example pump device of a fluid control system of a fluid-operated inflatable device.

FIG. 10B is a cross-sectional view of the example pump device shown in FIG. 10A, in an open position.

FIG. 11A is a schematic diagram of a roll of metal foil fabricated from a roll-milling process that causes a natural curvature in the foil of the roll.

FIG. 11B is a schematic diagram of a non-flat, unstressed diaphragm attached to a base plate.

FIGS. 12A and 12B are schematic cross-sectional diagrams of a system for creating a non-flat diaphragm in an unstressed state that can be used in the fluidic pumps described herein.

FIGS. 13A and 13B are schematic cross-sectional diagrams of another system for creating a non-flat diaphragm in an unstressed state that can be used in the fluidic pumps described herein.

FIGS. 14A and 14B are top and side schematic views, respectively, of a metal diaphragm being attached a metal base plate, where the thermal expansion of the metal materials is used to create a dome-shaped diaphragm on the base plate.

FIGS. 15A, 15B, and 15C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIG. 16 is a schematic end view of a filter foil.

FIGS. 17A, 17B, and 17C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIGS. 18-21 illustrate a filter member according to an embodiment.

FIGS. 22-29 illustrate filter members according to embodiments.

FIG. 30 is cross-sectional view of a valve device including a filter located at an end of a first fluid passageway and a filter located within a second fluid passageway.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

An implantable fluid-operated inflatable device may include a fluid control system. In some examples, the fluid control system includes at least one pump and/or at least one valve and/or at least one combined pump and valve device. In some examples, the components of the fluid control system control the flow of fluid between a fluid reservoir and an inflatable member of the implantable fluid-operated inflatable device, to provide for the inflation/pressurization and deflation/depressurization of the inflatable member. In some situations, the fluid control system may experience fluctuations in pressure, including spikes in pressure. For example, in some situations, the pumps and/or valves of the fluid control system may experience pressure fluctuations and/or spikes in pressure that exceed typical design pressures. In some situations, these types of pressure fluctuations, or spikes, may result in an unintended opening of a pump or valve, and/or unintended deflation/depressurization of the inflatable member, and/or unintended inflation/pressurization of the inflatable member. A fluid control system, in accordance with implementations described herein, includes a fluid control device including a check valve that prevents the unintentional opening of a fluid passageway in response to a spike in pressure experienced within the fluid control system. In some examples, the fluid control device is one of a pumping device, a valve device, or a combined pump and valve device. In some examples, the check valve is positioned in an outlet flow port of the fluid control device. In some examples, the check valve is positioned in an inlet flow port of the fluid control device. In some examples, the check valve includes a spiral check valve positioned in a fluid passageway of the fluid control device. In some examples, the check valve includes a spool shaped O-ring positioned in a fluid passageway of the fluid control device. In some examples, the check valve includes an umbrella valve positioned in a fluid passageway of the fluid control device. In some examples, a filter can be included in one or more pumps and/or valves to block the admission of particulate matter into the pumps and/or valves of the fluid control system.

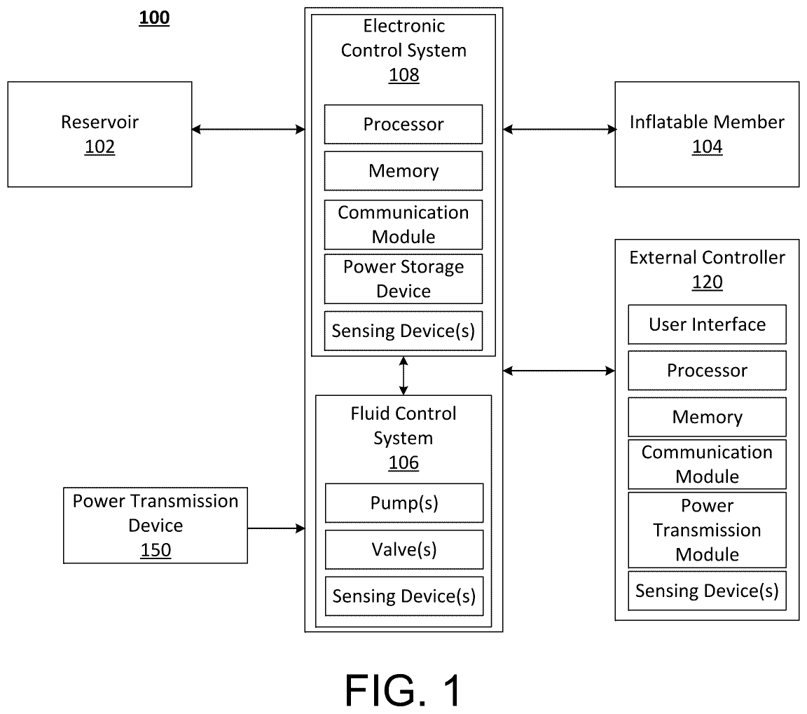

FIG. 1 is a block diagram of an example implantable fluid-operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidics components such as one or more pumps, one or more valves and the like configured to transfer fluid between the fluid reservoir 102 and the inflatable member 104. The fluid control system 106 can include one or more sensing devices that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid-operated inflatable device 100 and/or communication with one or more external device(s). In some examples, the electronic control system 108 includes components such as a processor, a memory, a communication module, a power storage device, or battery, sensing devices such as, for example an accelerometer, and other such components configured to provide for the operation and control of the implantable fluid-operated inflatable device 100. In some examples, the communication module of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation, and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in or incorporated into a housing. In some implementations, at least a portion of the electronic control system 108 is physically separate from the fluid control system 106. In some implementations, some modules of the electronic control system 108 are coupled to or incorporated into the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid-operated inflatable device 100. In some implementations, at least some aspects of the operation of the implantable fluid-operated inflatable device 100 may be manually controlled.

In some examples, electronic monitoring and control of the implantable fluid-operated inflatable device 100 may provide for improved patient control of the device, improved patient comfort, improved patient safety, and the like. In some examples, electronic monitoring and control of the implantable fluid-operated inflatable device 100 may afford the opportunity for tailoring of the operation of the inflatable device 100 by a physician without further surgical intervention. Fluidic architecture defining the flow and control of fluid through the implantable fluid-operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

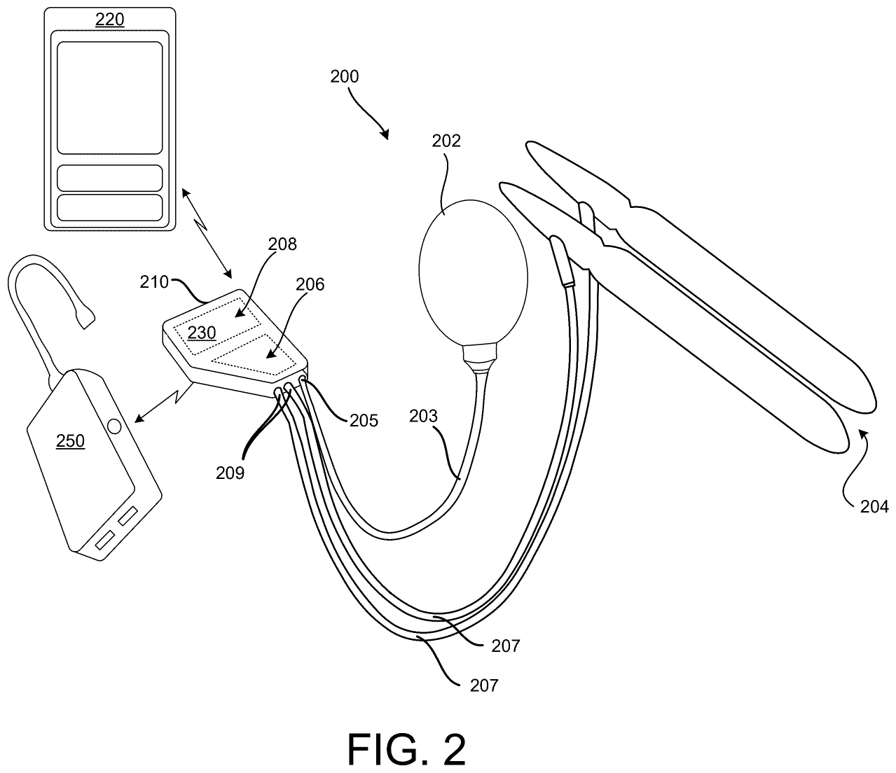

The example implantable fluid-operated inflatable device 100 may be representative of a number of different types of implantable fluid-operated devices. For example, the implantable fluid-operated inflatable device 100 shown in FIG. 1 may be representative of an inflatable penile prosthesis as shown in FIG. 2. In some implementations, the example implantable fluid-operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid-operated inflatable device 200 in the form of an example inflatable penile prosthesis is shown in FIG. 2. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2, the inflatable member 204 is in the form of a pair of inflatable cylinders. In the example shown in FIG. 2, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the example shown in FIG. 2, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 can be charged and/or recharged by a power transmission module of the external controller 220, and/or by a power transmission device 250, that is separate from the external controller 220.

The principles to be described herein are applicable to the example implantable fluid-operated inflatable device, in the form of the example inflatable penile prostheses shown in FIG. 2, and to other types of implantable fluid-operated inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. The example implantable fluid-operated inflatable device 200 shown in FIG. 2 includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid-operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control of the inflatable device 200, improved accuracy in operation of the inflatable device 200, improved patient comfort, improved patient safety, and the like. In some situations, this improved control and improved accuracy in the operation of the inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and/or valve devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices and/or combined pump and valve devices within a fluid circuit of the pump assembly to control the transfer fluid between the fluid reservoir and the inflatable member. In some examples, the pump assembly including the one or more pump devices and valve device(s) and/or combined pump and valve devices is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices and/or combined pump and valve devices include piezoelectric elements. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide for patient safety and device efficacy.

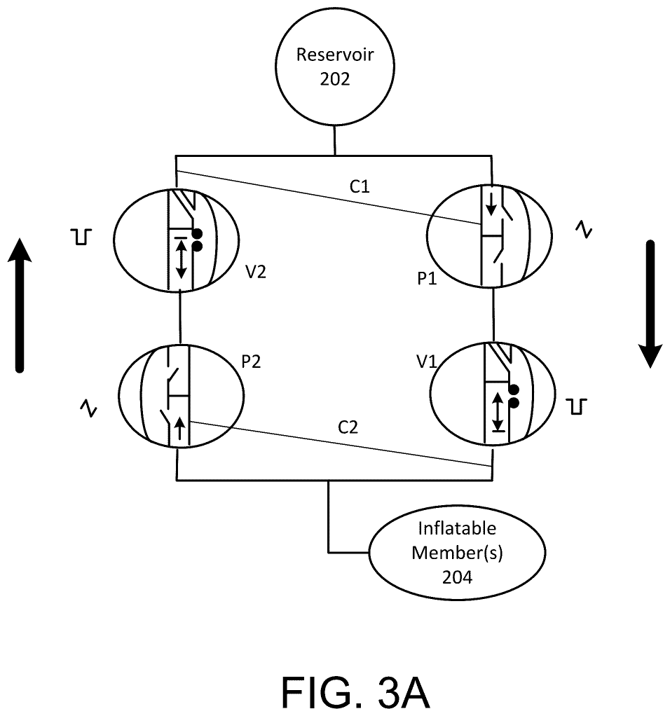

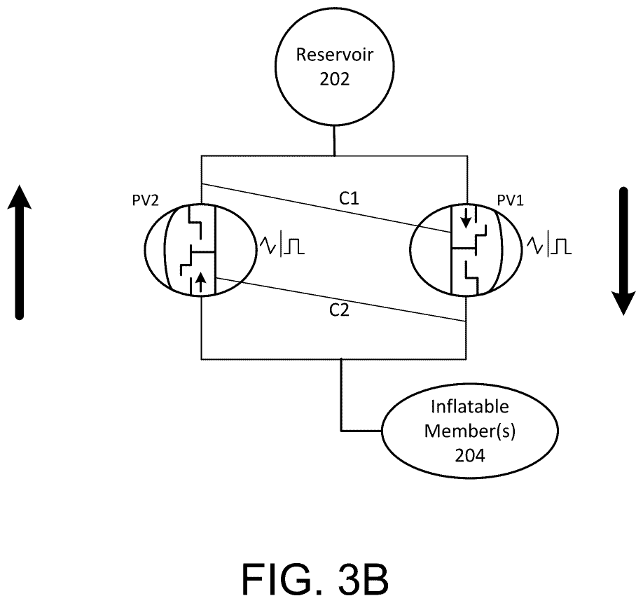

FIG. 3A is a schematic diagram of an example fluidic architecture for an implantable fluid-operated inflatable device, according to an aspect. FIG. 3B is a schematic diagram of an example fluidic architecture for an implantable fluid-operated inflatable device, according to an aspect. The fluidic architecture of an implantable fluid-operated inflatable device can include other arrangements of fluidic passageways, pump(s)/valve(s), pressure sensor(s) and other components than the examples shown in FIGS. 3A and 3B.

The example fluidic architecture shown in FIG. 3A includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3A includes a second pump P2 and a second valve V2 positioned in a second fluid passageway, between the inflatable member 204 and the reservoir 202, to control the flow of fluid from the inflatable member 204 to the reservoir 202. As shown in FIG. 3B, in some examples, the first pump and the first valve are included in a combination pump and valve device PV1 provided in the first fluid passageway, and the second pump and the second valve are included in a second combination pump and valve device PV2 provided in the second fluid passageway.

In example fluidic architecture shown in FIG. 3A, the first pump P1 and the first valve V1 operate to pump fluid from the reservoir 202 to the inflatable member 204 through the first fluid passageway to provide for inflation of the inflatable member 204, while the second valve V2 closes the second fluid passageway to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

In an optional example implementation, a conduit C1 can connect a section of the second fluid passageway that is downstream of pump P2 and valve V2 to a section of the first fluid passageway, for example, to an inlet portion of pump P1. Fluid flow through conduit C1 can flush fluid and material from out of the section of the first fluid passageway when fluid is pumped from the inflatable member 204 to the reservoir 202. In an optional example implementation, a conduit C2 can connect a section of the first fluid passageway that is downstream of pump P1 and valve V1 to a section of the second fluid passageway, for example, to an inlet portion of pump P2. Fluid flow through conduit C2 can flush fluid and material from out of the section of the second fluid passageway when fluid is pumped from the reservoir 202 to the inflatable member 204.

In the example arrangement shown in FIG. 3B, the first combined pump and valve device PV1 and the second combined pump and valve device PV2 may be operated in a first mode to inflate or pressurize the inflatable member 204 and in a second mode to deflate or depressurize the inflatable member 204. In the first mode of operation, the first combined pump and valve device PV1 convey fluid from the reservoir 202 to the inflatable member 204, while the second combined pump and valve device PV2 remains closed/inoperable to prevent flow of fluid from the inflatable member 204 towards the reservoir 202 to prevent deflation/depressurization. The first combined pump and valve device PV1 may remain operable to pump fluid to the inflatable member 204 until a desired pressure is achieved. The first combined pump and valve device PV1 may be closed once the desired pressure is achieved, to maintain the inflatable member 204 at the desired pressure/inflated state. In the second mode of operation, the second combined pump and valve device PV2 convey fluid from the inflatable member 204 to the reservoir 202, while the first combined pump and valve device PV1 remains closed/inoperable to prevent flow of fluid from the reservoir 202 towards the inflatable member 204 to prevent inflation/pressurization. The second combined pump and valve device PV2 may remain operable to pump fluid to the reservoir 202 until a desired pressure is achieved at the inflatable member 204. The second combined pump and valve device PV2 may be closed once the desired pressure is achieved, to maintain the inflatable member 204 at the desired pressure/in the deflated state.

In an optional example implementation, a conduit C1 can connect a section of the second fluid passageway that is downstream of pump P2 and valve V2 to a section of the first fluid passageway, for example, to an inlet portion of pump P1. Fluid flow through conduit C1 can flush fluid and material from out of the section of the first fluid passageway when fluid is pumped from the inflatable member 204 to the reservoir 202. In an optional example implementation, a conduit C2 can connect a section of the first fluid passageway that is downstream of pump P1 and valve V1 to a section of the second fluid passageway, for example, to an inlet portion of pump P2. Fluid flow through conduit C2 can flush fluid and material from out of the section of the second fluid passageway when fluid is pumped from the reservoir 202 to the inflatable member 204.

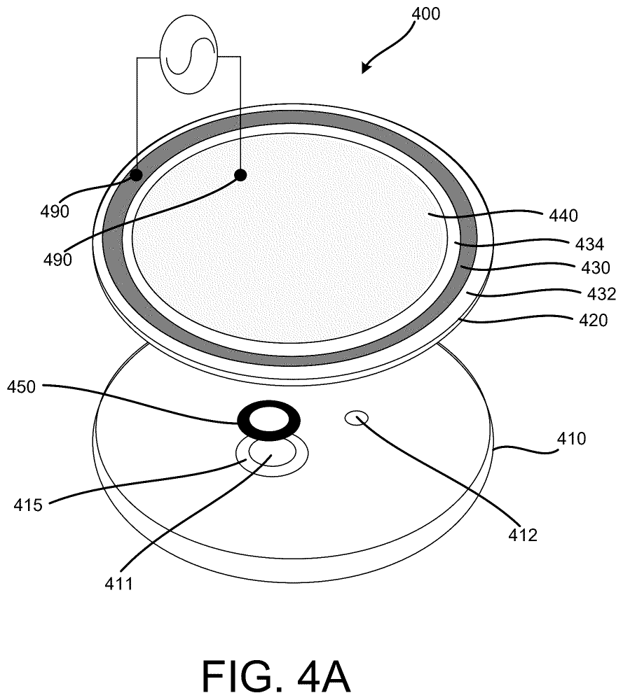

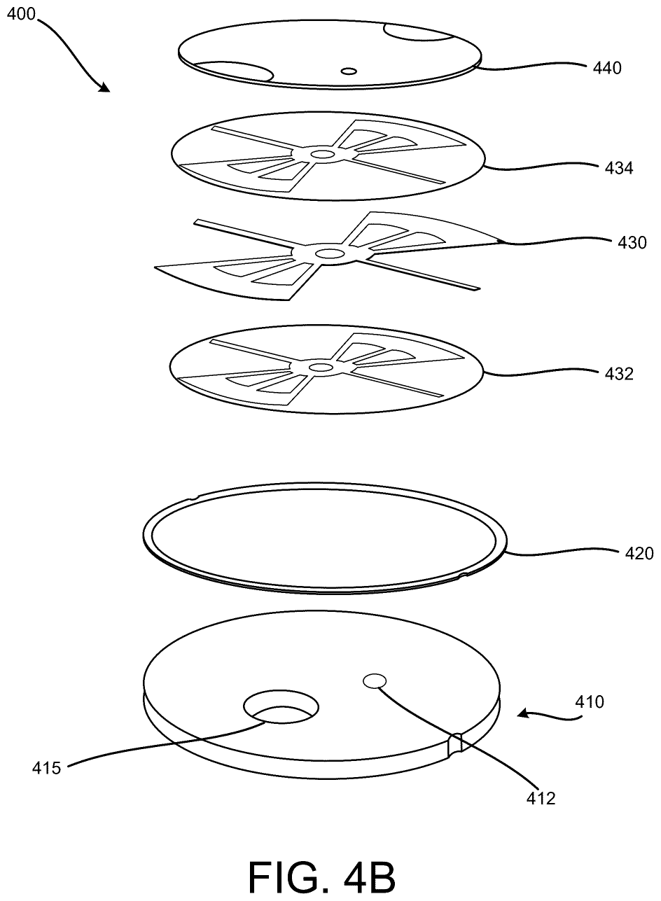

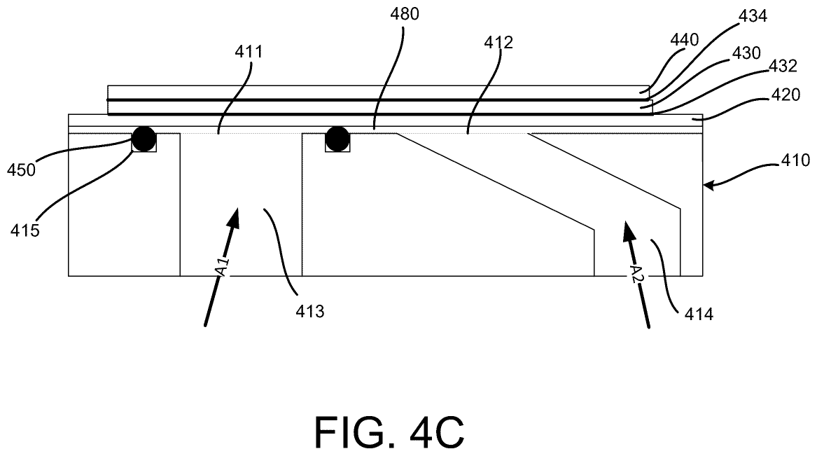

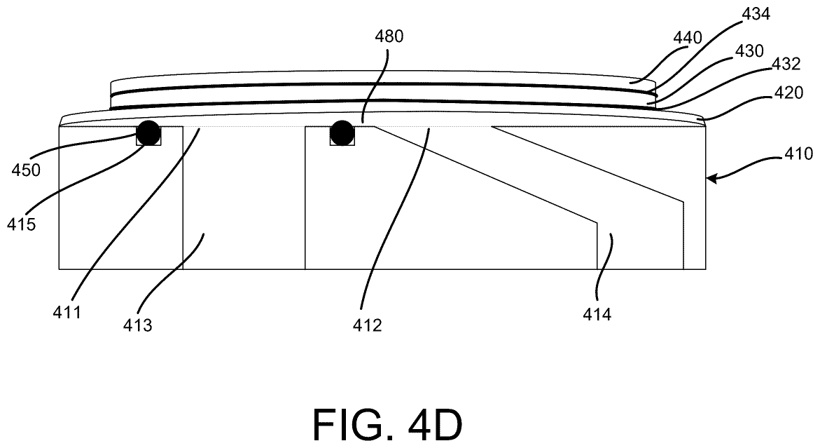

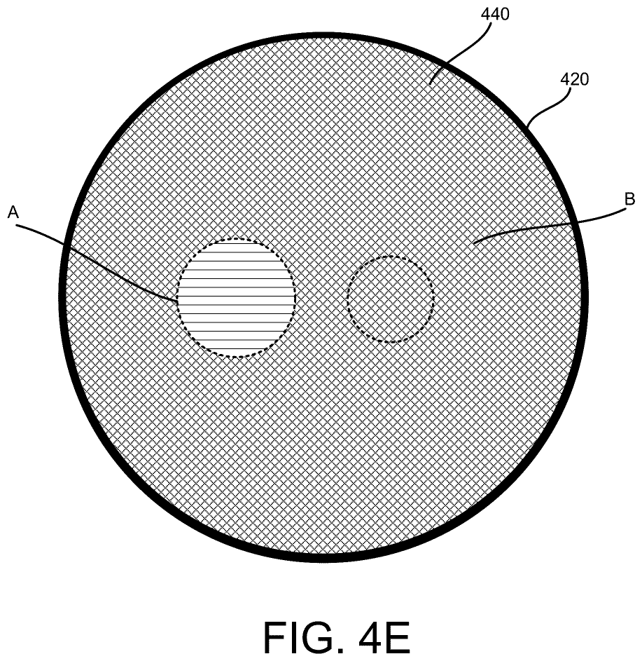

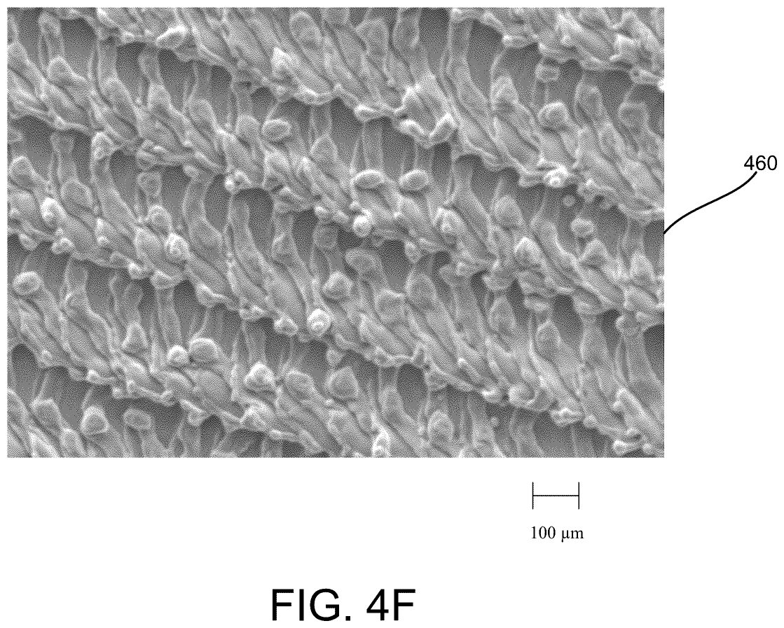

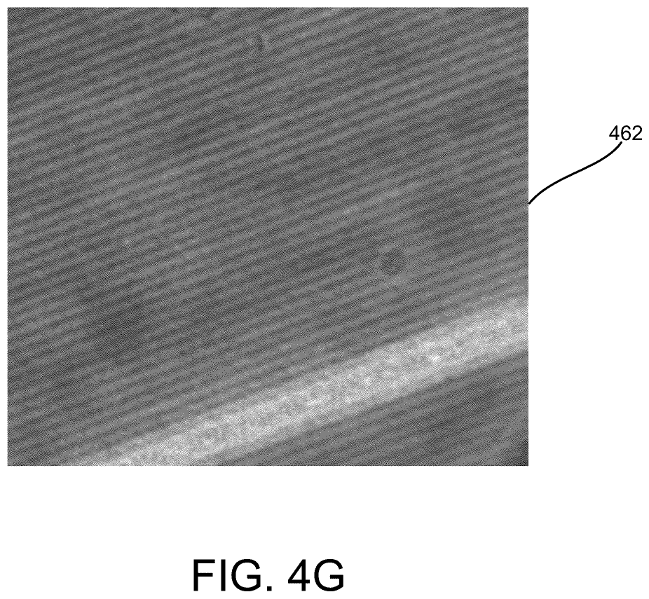

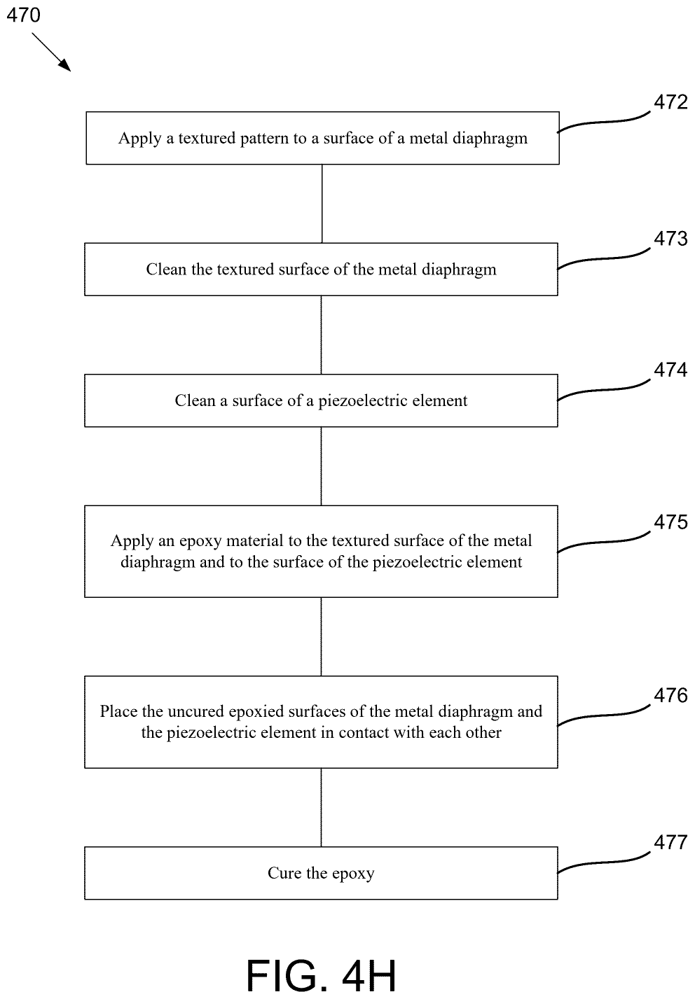

FIG. 4A is a partially exploded perspective view of an example valve device 400. FIG. 4B is an exploded perspective view of the example valve device 400. FIGS. 4C and 4D are cross-sectional views of the example valve device 400 shown in FIG. 4A, in an assembled state. FIG. 4E is a schematic top view of the example valve device 400. The example valve device 400 shown in FIGS. 4A-4E is an example of a fluid control device, or a fluidic component, included in the fluid control system 206 of the example electronically controlled fluid manifold 230 described above.

In the example arrangement shown in FIGS. 4A-4E, the example valve device 400 includes a base plate 410 defining a base portion of the valve device 400. A diaphragm 420 is positioned on the base plate 410. A piezoelectric element 440 is positioned on the diaphragm 420, with an isolation layer 430 positioned between the diaphragm 420 and the piezoelectric element 440. The piezoelectric element can be electrically powered (e.g., by a battery of in the implantable fluid-operated inflatable device 100) to drive the diaphragm 420 to open and close the valve device 400. The diaphragm 420 can include a thin metal foil, whose shape can be repeatably deformed in response to movement by the piezoelectric element 440. In some implementations, the diaphragm 420 can include titanium material. In some implementations, the diaphragm 420 can include gold material. In some implementations, the diaphragm 420 can include stainless steel material or other alloys. In some implementations, the isolation layer 430 can include a polyamide material that has a high resistivity, for example, a resistivity greater than 10¹³ Ohm-cm to provide electrical isolation between the piezoelectric element 440 and the diaphragm 420.

In some examples, an epoxy layer 432 provides for the coupling of the isolation layer 430 and the diaphragm 420. In some examples, an epoxy layer 434 provides for the coupling of the piezoelectric element 440 and the isolation layer 430, and the epoxy layers 432, 434 together provide for the coupling of the piezoelectric element 440 to the diaphragm 420. In some implementations, the epoxy layers 432, 434 are not distinct but are part of one epoxy layer. The epoxy layers 432, 434 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 490 are arranged on the example valve device 400. In the example shown in FIG. 4A, the example valve device 400 includes a pair of electrodes 490 coupled between the isolation layer 430 and the piezoelectric element 440. Application of a voltage to the piezoelectric element 440 causes a deflection or deformation of the piezoelectric element 440 and a corresponding deflection or deformation of the diaphragm 420 coupled thereto.

In the example arrangement shown in FIGS. 4A-4D, a fluid chamber 480 is defined between the base plate 410 and the diaphragm 420. The base plate 410 includes a first opening 411 that provides for communication between a first fluid passageway 413 and the fluid chamber 480. The base plate 410 includes a second opening 412 that provides for communication between a second fluid passageway 414 and the fluid chamber 480. In the example arrangement shown in FIGS. 4A-4D, the base plate 410 includes a recess 415 surrounding the first opening 411, with a seal 450, in the form of an O-ring in the example shown in FIGS. 4A-4D, fitted in the recess 415. In some examples, a top portion of the seal 450 is pressed against the diaphragm 420 in the closed position of the valve device 400, as shown in FIG. 4C to close off the chamber 480 and inhibit the flow of fluid through the example valve device 400, between the first fluid passageway 413 and the second fluid passageway 414 via the chamber 480. In some examples, in which the valve device 400 does not include a seal 450, the diaphragm 420 is seated against the base plate 410 to close off the chamber 480 and inhibit the flow of fluid through the valve device 400. In the open position of the example valve device 400, the base plate 410 and the top portion of the seal 450 are separated, or spaced apart from, the diaphragm 420 due to the deflection of the diaphragm 420. This positioning of the seal 450 and the base plate 410 relative to the diaphragm 420 opens the chamber 480 and allows fluid to flow through the example valve device 400, between the first fluid passageway 413 and the second fluid passageway 414 via the fluid chamber 480.

In some situations, the fluctuations, or spikes in pressure may be greater than a pressure applied by the piezoelectric element 440 (and isolation layer 430 and diaphragm 420 coupled thereto) on the seal 450 and/or the base plate 410 to maintain a closed state of the valve device 400. For example, pressure applied in the direction of the arrow A1 (via the first fluid passageway 413 and first opening 411) and/or in the direction of the arrow A2 (via the second fluid passageway 414 and second opening 412) may exert a pressure on the piezoelectric element 440/isolation layer 430/diaphragm 420 that is greater than the closing force exerted on the seal 450 and/or base plate 410 by the piezoelectric element 440/isolation layer 430/diaphragm 420. In some examples, the force A1 may exert a pressure on the piezoelectric element 440/isolation layer 430/diaphragm 420 in a pressure area A shown in FIG. 4E. In some examples, the force A2 may exert a pressure on the piezoelectric element 440/isolation layer 430/diaphragm 420 in a pressure area B shown in FIG. 4E.

Forces, or pressure exerted on the piezoelectric element 440/isolation layer 430/diaphragm 420 in this manner can cause an unintentional opening of the valve device 400. This unintentional opening of the valve device 400 may, in turn, cause unintentional deflation/depressurization of the inflatable member 204, or unintentional inflation/pressurization of the inflatable member 204, depending on a direction of flow through the valve device 400 (or other fluid control device of the fluid control system 206).

In some implementations, a fluid control device of the fluid control system 206, such as the example valve device 400, includes a check valve, or a one-way valve, positioned in a fluid passageway of the fluid control device. In some examples, the check valve is positioned in a portion of the fluid passageway so as to inhibit the unintended flow of fluid through the fluid control device in the event of a fluctuation, or spike in pressure. In some examples, the check valve is positioned in the fluid passageway so as to counteract a back pressure that would otherwise overcome the closing pressure and cause unintentional flow through the device. In some examples, a check valve is positioned at the second opening 412 in the base plate 410, defining an interface between the second fluid passageway 414 and the chamber 480 of the example valve device 400. In some examples, a check valve is positioned at the first opening 411 in the base plate 410, defining an interface between the first fluid passageway 413 and the chamber 480 of the example valve device 400.

Because the valve device 400 must operate reliably over many thousands or hundreds of thousands of opening/closing cycles (e.g., cycles of the diaphragm 420 moving between an open position and a closed position), a strong and durable connection between the piezoelectric element 440 and the diaphragm 420 is desirable. Therefore, a strong and durable bond between the epoxy 434 and the piezoelectric element 440 and the epoxy 434 and between the epoxy 432 and the diaphragm 420 is important. In particular, when the diaphragm 420 includes titanium, because it can be difficult to form a strong adhesive bond between epoxy and titanium, as compared to the bond strength between epoxy and some other materials, the bond strength and durability of the epoxy/titanium connection is important. To enhance the strength and durability of the bond between the epoxy layer 432 and the diaphragm 420, a surface of the diaphragm can be textured before the application of the epoxy 432 to the diaphragm 420. By texturing the surface, or by increasing the roughness of the surface, the total surface area of the diaphragm 420 with which the epoxy bonds can be increased, and the strength and durability of the connection between the epoxy 432 and the metal diaphragm 420 can be increased.

The surface of the diaphragm 420 can be textured using a number of different techniques. For example, a random or erratic texture pattern can be applied to the surface by bead blasting with particles such as, for example, sand or aluminum oxide particles. In another example, a texture pattern can be applied to the surface through a chemical etching process. In another example, a texture pattern can be applied to the surface through a plasma etching process. In another example, a predetermined texture pattern can be applied to that surface through a laser etching or ablation process. Combinations of different techniques can be used to apply a texture to the surface.

FIG. 4F is a microscopic image 460 of a surface of the diaphragm 420 after an erratic texture pattern has been applied to the surface. By imparting the erratic texture pattern to the surface of the diaphragm 420 (e.g., through bead blasting, chemical etching, plasma etching, etc.), surface features less than 100 microns can be embedded in the surface of the diaphragm 420. The erratic texture pattern imparted to the surface can enhance the adhesion of the epoxy to the surface due to the increased surface area of the texture surface, which offers more bonding contact points for the epoxy to the surface then does a smooth metal surface.

In addition, the epoxy/metal interface can be more resistant to delamination of the epoxy layer from the metal diaphragm 420 when the surface of the diaphragm has been textured prior to application of epoxy to the diaphragm, as compared with a smooth surface of the metal diaphragm. The increased resistance to delamination can be brought about because surface features of the textured surface can provide locations at which initial microscopic cracks, fissures, or faults in the epoxy/metal interface stop further propagation of initial microscopic cracks, fissures, or defaults.

FIG. 4G is a microscopic image 462 of a surface of the diaphragm 420 after a periodic texture pattern has been applied to the surface. As with the erratic pattern shown in image 460 of FIG. 4F, by imparting the period texture pattern to the surface of the diaphragm 420 (e.g., laser etching, etc.), surface features on the order of 100 microns or smaller can be embedded in the surface of the diaphragm 420. The periodic texture pattern imparted to the surface can enhance the adhesion of the epoxy to the surface due to the increased surface area of the texture surface, which offers more bonding contact points for the epoxy to the surface then does a smooth metal surface.

The microscopic image 462 of FIG. 4G shows a periodic pattern of laser etched parallel lines, or ridges, of material in the surface of the metal diaphragm 420, but other patterns also are possible. For example, multidirectional patterns that are periodic in more than one dimension can provide increased shear strength and resistance to delamination in a plurality of directions across the surface of the diaphragm 420. In some implementations, a laser-etched texture pattern can include parallel lines of ridges in the surface. In some implementations, a laser-etched texture pattern can include curved or wavy lines of ridges in the surface. In some implementations, a laser-etched texture pattern can include ridges having differing heights in the surface. In some implementations, a laser-etched texture pattern can include ridges in the surface, where the ridges have different periodicities at different locations on the surface. In some implementations, a laser-etched texture pattern can include features other than lines, such as, for example, dimples, bumps, geometric shapes, etc. Combinations of different patterns described herein also can be applied to the surface of the diaphragm. Such multidimensional patterns can reduce or inhibit stress concentrations and defect propagation in the epoxy/metal interface between the epoxy layer 432 and the metal diaphragm 420.

A texture pattern on the surface of the diaphragm 420 can be further treated before application of the epoxy layer 432 to the surface of the diaphragm. For example, the surface can be cleaned with one or more chemical solvents (e.g., acetone, ethanol, nitric acid, hydrofluoric acid) and/or treated with a plasma etching process prior to application of the epoxy layer 432 to the diaphragm 420. A surface of the piezoelectric element 440 also can be cleaned with one or more chemical solvents (e.g., acetone, ethanol, nitric acid, hydrofluoric acid) and/or treated with a plasma etching process prior to application of the epoxy layer 434 to the piezoelectric element 440.

FIG. 4H is a flowchart of a process 470 for assembling components of a valve device 400 for use in an implantable device. The process 470 can include applying a texture pattern to a surface of a metal diaphragm (472). In some implementations, applying the texture pattern to the surface can include one or more forms of surface modification, such as, for example, laser etching, bead blasting, chemical etching, plasma etching, and the like. The process 470 can include cleaning the textured surface of the metal diaphragm (473). In some implementations, cleaning the textured surface can include one or more of chemical cleaning, abrasive cleaning, plasma cleaning, and the like. The process 470 can include cleaning a surface of a piezoelectric element (474), and, in some implementations, the cleaning can include one or more of chemical cleaning, abrasive cleaning, plasma cleaning, and the like. The process 470 can include applying an epoxy material to the textured surface of the metal diaphragm and to the surface of the piezoelectric element (475), placing the uncured epoxied surfaces of the metal diaphragm and the piezoelectric element in contact with each other (476), and curing the epoxy (477). In some implementations, before the diaphragm 420 is placed in contact with the piezoelectric element 440, a voltage can be placed across the electrodes 490 attached to the piezoelectric element 440 to configure the piezoelectric element 440 in the domed configuration that is assumes when the fluid chamber is in the open position (See FIG. 4D). Then, the diaphragm can be placed in contact with the piezoelectric element while the piezoelectric element 440 is in its domed configuration, and the epoxy can be cured when the piezoelectric element and the diaphragm 420 are in the domed configuration, which can reduce stress on the adhesive bond between the diaphragm 420 and the piezoelectric element 440.

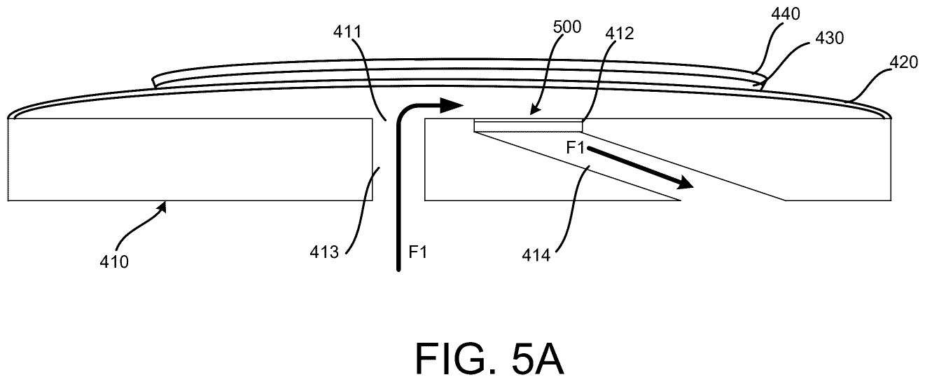

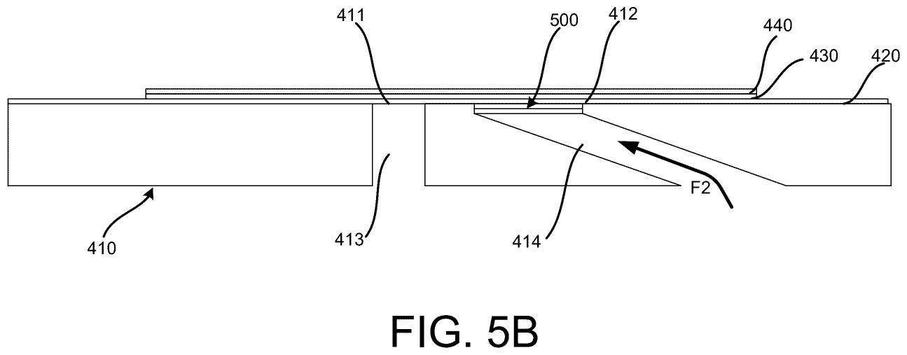

FIGS. 5A and 5B are cross-sectional views of the example valve device 400 shown in FIGS. 4A-4E, including an example flow control device 500 positioned in one of the fluid passageways of the example valve device 400.

FIG. 5A illustrates an example in which the valve device 400 is open, allowing fluid to flow in the direction of the arrows F1, through the first fluid passageway 413, into the chamber 480, and out of the valve device 400 through the second fluid passageway 414. The example shown in FIG. 5A may illustrate an open position of the valve device 400 that allows fluid to flow, for example, from the reservoir 202 to the inflatable member 204 to provide for inflation/pressurization of the inflatable member 204.

In the example arrangement shown in FIGS. 5A and 5B, the example flow control device 500 is positioned at the second opening 412 formed in the base plate 410, the second opening 412 providing for fluid communication between the fluid chamber 480 and the second fluid passageway 414. In some examples, the flow control device 500 is a check valve, or a one-way valve, which allows for flow in one direction (in this example, in the direction of the arrows F1), while inhibiting flow in the opposite direction.

FIG. 5B illustrates the closed position of the valve device 400, in which the flow of fluid through the valve device 400 is blocked. In some examples, the closed position shown in FIG. 5B may maintain an inflation pressure of the inflatable member 204. As described above, in some situations, pressure fluctuations and/or pressure spikes may exert a force, or pressure on the valve device 400 in the closed position. FIG. 5B illustrates a pressure spike, or a back pressure, exerted in the direction of the arrow F2. In the example described above with respect to FIGS. 4A-4E, this type of pressure spike, or back pressure exerted on the diaphragm 420/piezoelectric element 440 could cause an unintentional opening of the valve device 400, and an unintentional deflation/depressurization of the inflatable member 204. In the example shown in FIG. 5B, the flow control device 500 (positioned at the second opening 412, between the second fluid passageway 414 and the fluid chamber 480), for example, in the form of a check valve or a one-way valve, remains in the closed position in response to the pressure spike/back pressure/flow of fluid in the direction of the arrow F2. Thus, the positioning of the flow control device 500 at the second opening 412, allowing flow in a first direction, i.e., the direction of the arrows F1, while blocking flow in a second direction, i.e., the direction of the arrow F2, maintains the closed state of the valve device 400, even in response to fluctuation in pressure, or pressure spike, or back pressure.

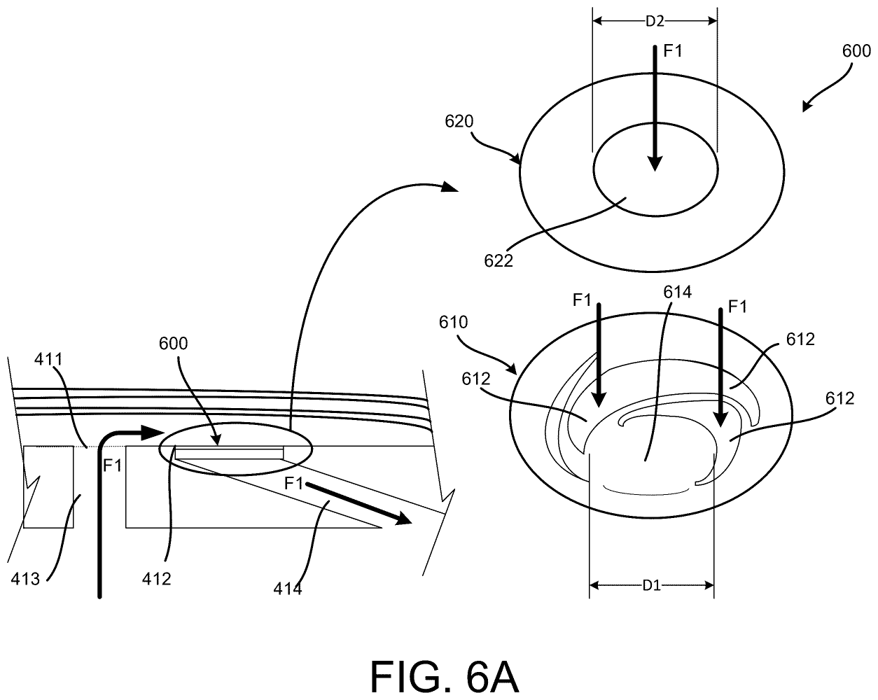

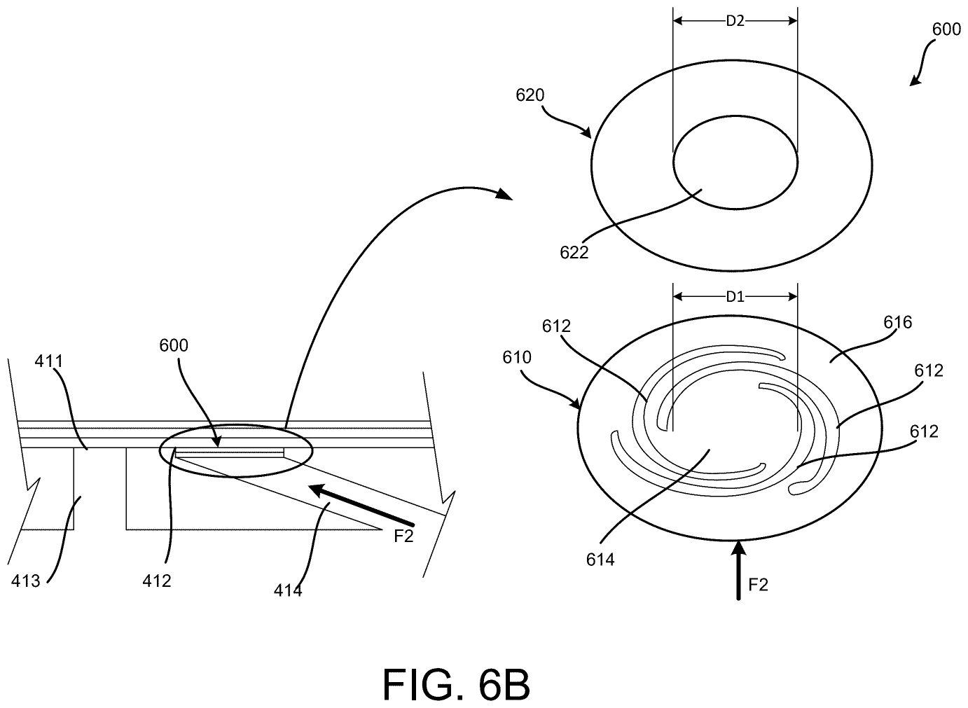

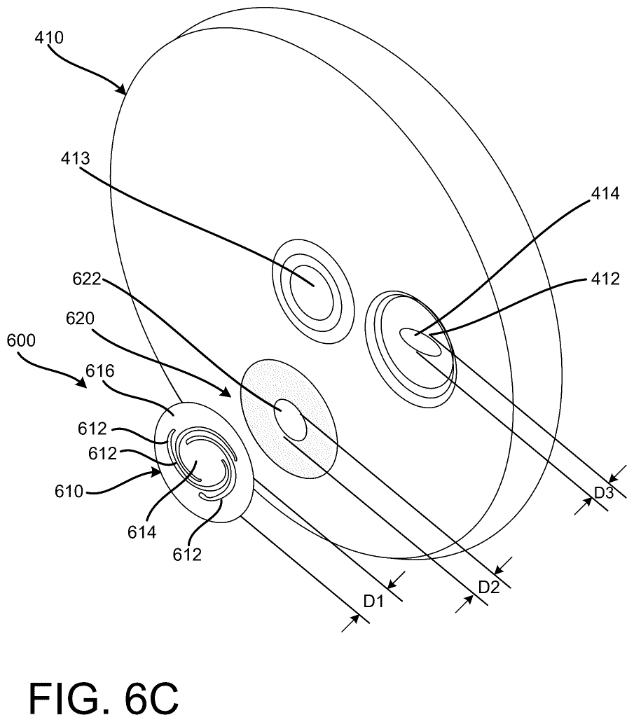

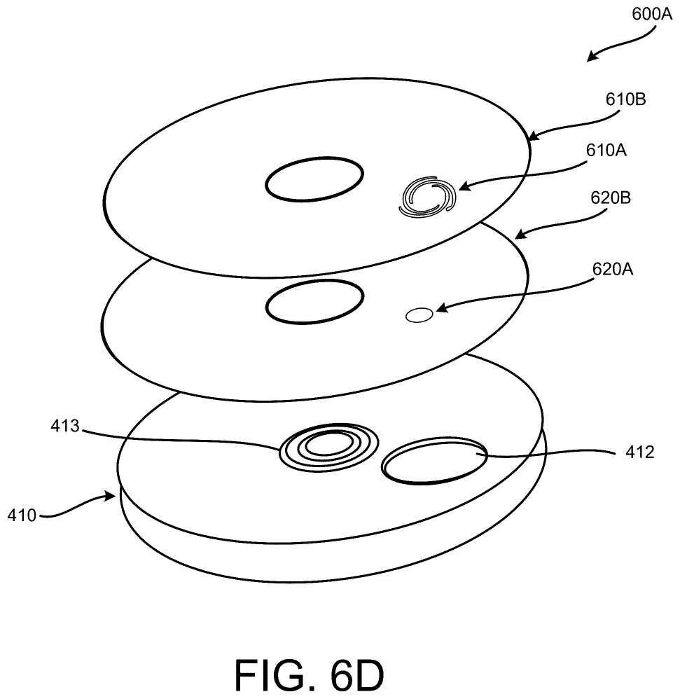

FIGS. 6A-6C illustrate an example auxiliary flow control device 600 that can be incorporated into the example valve device 400, for example, in the manner described above with respect to FIGS. 5A and 5B. In particular, the example auxiliary flow control device 600 may be a check valve, or a one-way valve, which provides for flow in one direction, and that inhibits flow in a second direction.

FIG. 6A illustrates the example flow control device 600 in the example valve device 400, in the open position. FIG. 6B illustrates the example flow control device 600 in the example valve device 400, in the closed position. FIG. 6C is an exploded perspective view of the example valve device 400 relative to the base plate 410 of the example valve device 400.

The example flow control device 600 shown in FIGS. 6A and 6B includes a spring check valve 610 positioned against a foil 620. In the open position of the flow control device 600 (corresponding, for example, to the open position of the valve device 400 shown in FIGS. 4D and 5A), fluid can flow in the direction of the arrows F1, through the flow control device 600. That is, a pressure of the fluid flowing from the fluid chamber 480 through the second opening 412 in the base plate 410 and an opening 622 in the foil 620 (for example, an opening 622 aligned with the second opening 412 in the base plate 410) exerts a pressure on a disc portion 614 of the spring check valve 610. The pressure, or force exerted on the disc portion 614 of the spring check valve 610 moves the disc portion 614 in the direction of the arrows F1. This movement of the disc portion 614 separates the disc portion 614 from a rim portion 616 of the spring check valve 610, so that fluid can flow through openings defined by slots 612 formed in the spring check valve 610 in the open, or expanded position shown in FIG. 6A.

In the closed position of the flow control device 600 (corresponding, for example, to the closed position of the valve device 400 shown in FIGS. 4C and 5B), the flow of fluid from the fluid chamber 480 into the second fluid passageway 414 is blocked by the position of the diaphragm 420 (and isolation layer 430/piezoelectric element 440) extending across the second opening 412 in the base plate 410. In the event of a spike in pressure, or force, or back pressure in the direction of the arrow F2, the flow control device 600 may maintain a closed position, or a closed state of the valve device 400. That is, the flow control device 600, in the form of a check valve or a one-way valve positioned in the second opening 412 in the base plate 410, may remain closed, even in the event of a spike in pressure, or back pressure, thus maintaining the closed state of the valve device 400.

For example, as shown in FIG. 6B, a dimension, for example, a diameter D1 of the disc portion 614 of the spring check valve 610 may be greater than a corresponding dimension, for example, a diameter D2 of the opening 622 of the foil 620. Thus, an overlap, or interference of an outer peripheral portion of the disc portion 614 of the spring check valve 610 with an inner peripheral portion of the opening 622 in the foil 620, thus restricting movement of the disc portion 614 of the spring check valve 610 through the opening 622 and closing the flow control device 600 and restricting flow from the second fluid passageway 414 into the fluid chamber 480.

The example flow control device 600 has been described with respect to the spring check valve 610 in combination with the foil 620 in FIGS. 6A-6C. The principles described can be similarly applied to the use of the spring check valve 610, without the foil 620, positioned in the second opening 412 in the base plate 410. That is, in a configuration of the flow control device 600 including just the spring check valve 610, movement of the disc portion 614 of the spring check valve 610 in the direction of the arrow F2 may be restricted by a position of the disc portion 614 against the diaphragm 420. In some examples, the diameter D1 of the disc portion 614 of the spring check valve 610 may be greater than a corresponding dimension D3 of a corresponding portion of the second opening 412 in which the flow control device 600 is installed. In this case, an overlap, or interference of the outer peripheral portion of the disc portion 614 of the spring check valve 610 with an inner peripheral portion of the second opening 412 in the base plate 410, thus restricting movement of the disc portion 614 of the spring check valve 610 through the second opening 412, thus maintaining the closed position of the flow control device 600.

FIG. 6D is an exploded perspective view, illustrating an example auxiliary flow control device 600A that can be incorporated into the example valve device 400. In particular, the example auxiliary flow control device 600A may include check valve, or a one-way valve, which provides for flow in one direction, and that inhibits flow in a second direction.

In the example arrangement shown in FIG. 6D, the example flow control device 600A includes a spring check valve 610A incorporated into a spring plate 610B, and a foil 620A incorporated into a foil plate 620B, with the foil plate 620B positioned between the spring plate 610B and the base plate 410 of the example valve device 400. The spring check valve 610A incorporated into the spring plate 610B, and the foil 620A incorporated into the foil plate 620B, may function similarly to the spring check valve 610 and foil 620 described above with respect to FIGS. 6A-6C, providing for resistance to spikes in pressure, or backpressure, during operation of the example valve device 400, particularly in the closed state of the example valve device 400. Similarly, in some examples, the spring check valve 610A incorporated into the spring plate 610B may be operable without the foil plate 620B to provide for resistance to spikes in pressure, or backpressure, during operation of the example valve device 400, particularly in the closed state of the example valve device 400.

In the examples presented above with respect to FIGS. 6A-6D, operation of the example valve device 400 including the example flow control device 600 positioned in the second opening 412 of the base plate 410 is described in an example arrangement in which fluid flows into the example valve device 400 via the first fluid passageway 413, and out of the example valve device 400 via the second fluid passageway 414, to, for example, provide for the flow of fluid from the reservoir 202 to the inflatable member 204, simply for purposes of discussion and illustration. The principles described above are similarly applicable to an arrangement in which the flow control device 600 is positioned in the first opening 411/first fluid passageway 413 (not explicitly shown in FIGS. 6A-6D), and in which fluid flows into the example valve device 400 via the second fluid passageway 414, and out of the example valve device 400 via the first fluid passageway 413 to, for example, provide for the flow of fluid from the inflatable member 204 to the reservoir 202. The principles described above are similarly applicable to an arrangement in which the flow control device 600 is positioned in the second opening 412/second fluid passageway 414, with spring 610/spring plate 610B positioned between the foil 620/foil plate 620B and the base plate 410 (not explicitly shown in FIGS. 6A-6D), and in which fluid flows into the example valve device 400 via the second fluid passageway 414, and out of the example valve device 400 via the first fluid passageway 413 to, for example, provide for the flow of fluid from the inflatable member 204 to the reservoir 202.

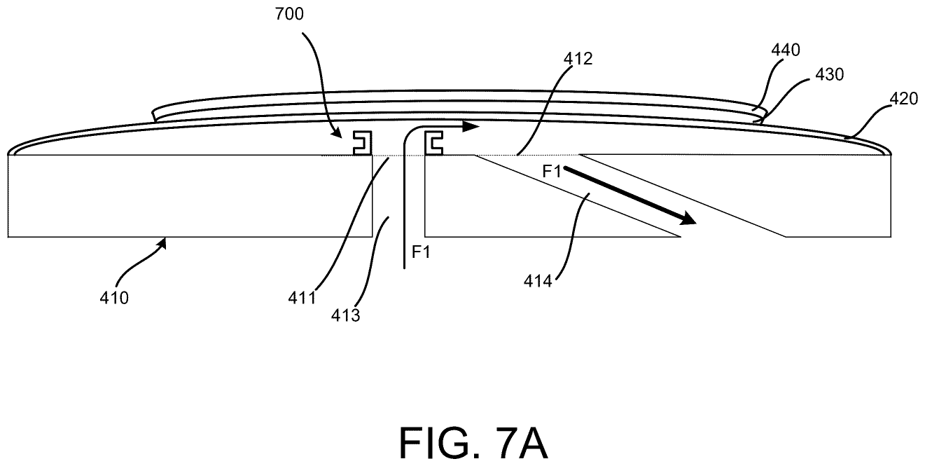

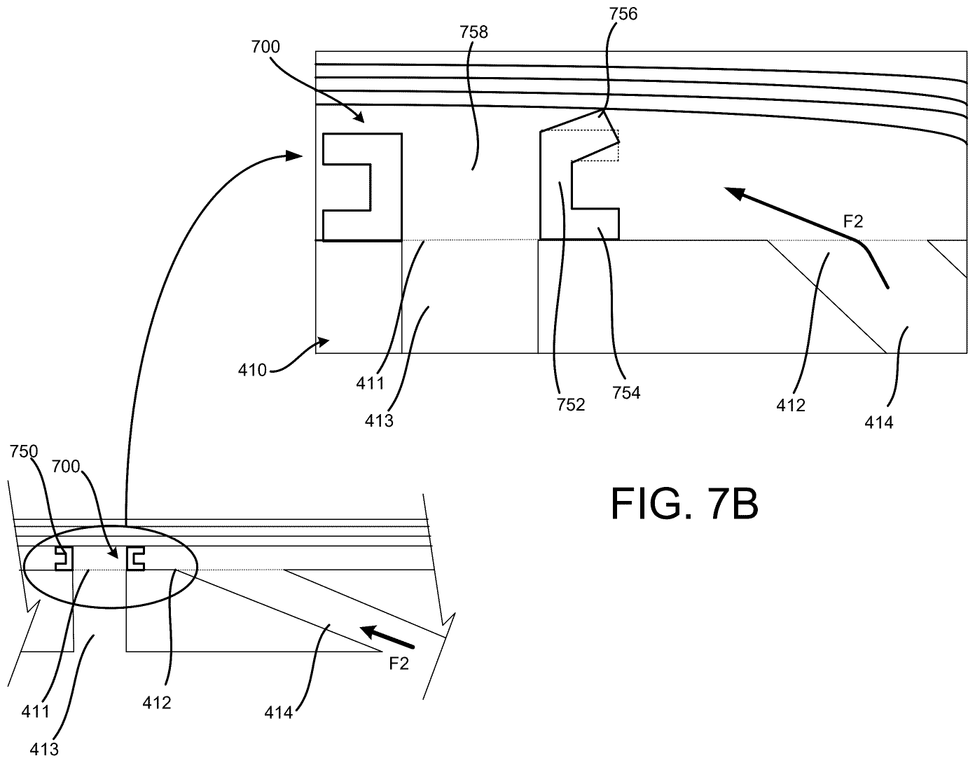

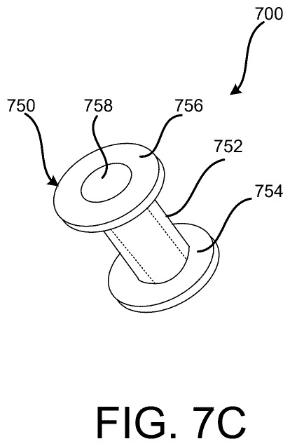

FIGS. 7A-7C illustrate an example auxiliary flow control device 700 that can be incorporated into the example valve device 400. In the example arrangement shown in FIGS. 7A-7C, the example auxiliary flow control device 700 is in the form of a seal 750 positioned at the first opening 411 formed in the base plate 410, at the interface between the first fluid passageway 413 and the fluid chamber 480 of the example valve device 400. The example flow control device 700 may allow for flow in one direction and inhibit flow in a second direction.

FIG. 7A illustrates the example flow control device 700 in the example valve device 400, in the open position. FIG. 7B illustrates the example flow control device 700 in the example valve device 400, in the closed position. FIG. 7C is a perspective view of the example flow control device 700, including features of a seal 750 defining the flow control device 700.

The seal 750 includes a body portion 752, with a first flange portion 754 extending outward, for example radially outward, from a first end portion of the body portion 752, and a second flange portion 756 extending outward, for example, radially outward, from a second end portion of the body portion 752. An opening 758 is defined in the body portion 752, extending through the seal 750. In some examples, the seal 750 is made of an elastomer material, or a compliant material, which allows for some amount of flex or movement in response to the application of force or pressure.

In the arrangement shown in FIGS. 7A and 7B, the first flange portion 754 of the seal 750 is fixed to the base plate 410, at a position corresponding to the first opening 411/first fluid passageway 413. The opening 758 in the seal 750 is aligned with the first opening 411/first fluid passageway 413 so that, in the open state of the valve device 400, fluid from the first fluid passageway 413 can flow through the first opening 411, through the opening 758 of the seal 750, through the fluid chamber 480, and out through the second opening 412/second fluid passageway 414 in the direction of the arrows F1, as shown in FIG. 7A.

In the closed position of the valve device 400 shown in FIG. 7B, the diaphragm 420 is positioned against, for example, pressed against, the second flange portion 756 of the seal 750. In this arrangement, the second flange portion 756 defines a sealing portion of the seal 750, with the pressure exerted on the second flange portion 756 by the diaphragm 420 forming a seal that blocks or inhibits the flow of fluid. That is, in the example arrangement shown in FIG. 7B, flow of fluid from the first fluid passageway 413 into and through the fluid chamber 480 is blocked or inhibited by the positioning of the diaphragm 420 against the second flange portion 756, and the seal formed therebetween.

In some examples, a spike in pressure, or force, or back pressure in the direction of the arrow F2 may cause the diaphragm 420 (and piezoelectric element 440/isolation layer 430) to deflect or deform, unintentionally opening a portion of the fluid chamber 480 with the valve device 400 in the closed state, as shown in the enlarged portion of FIG. 7B. In response to this spike in pressure, or force, or back pressure and corresponding flow of fluid in the direction of the arrow F2, while the valve device 400 is in the closed position, at least a portion of the seal 750 may deflect, or deform, to maintain the sealed condition of the fluid chamber 480, and restrict flow through the fluid chamber 480 in the closed state of the valve device 400, as shown in the enlarged portion of FIG. 7B. In particular, in the example shown in FIG. 7B, the force of the fluid exerted on the seal 750 in the direction of the arrow F2 causes the second flange portion 756 to deflect from an at rest position (shown in dashed lines) to a deflected position. This deflection maintains the seal between the flow control device 700 including the seal 750 and the diaphragm 420 and maintains the closed state of the valve device 400. Thus, the flow control device 700, in the form of the seal 750 positioned at the first opening 411 in the base plate 410, may deform or deflect to maintain a closed state of the fluid chamber 480 and a corresponding closed state of the valve device 400, even in the event of a fluctuation or spike in pressure, or back pressure.

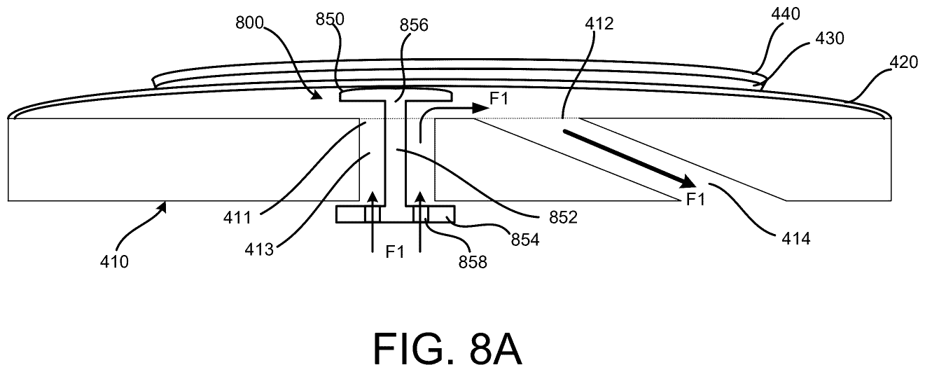

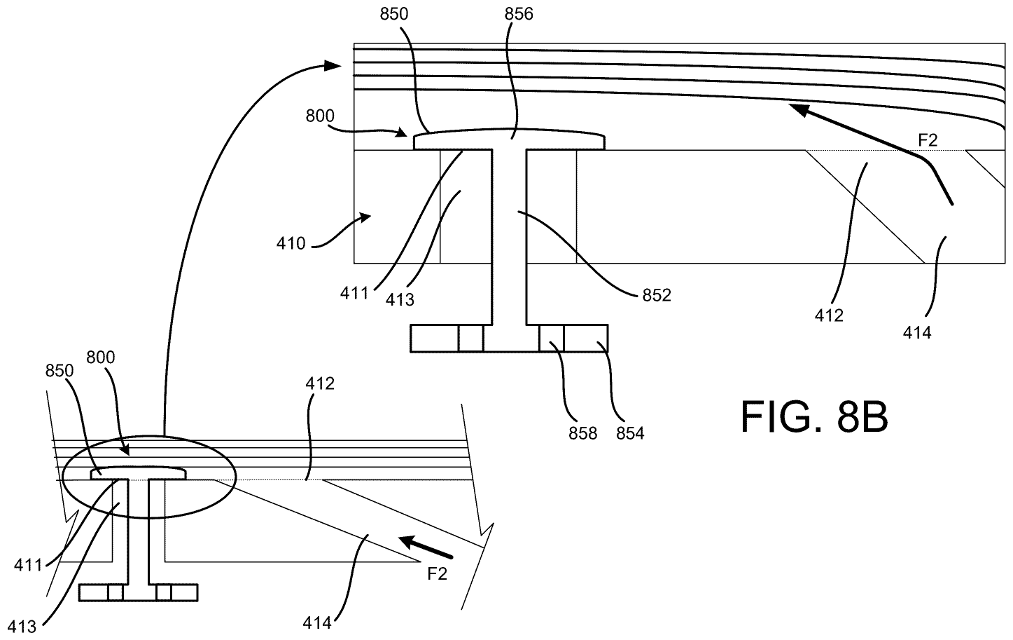

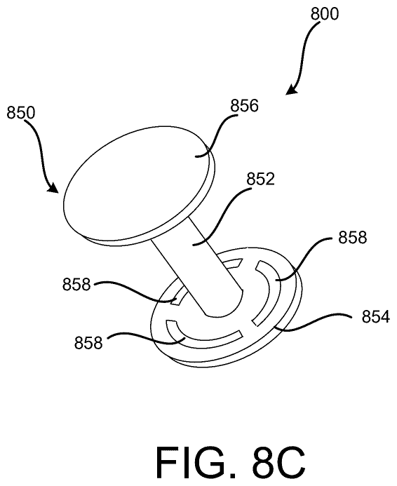

FIGS. 8A-8C illustrate an example auxiliary flow control device 800 that can be incorporated into the example valve device 400. In the example arrangement shown in FIGS. 8A-8C, the example auxiliary flow control device 800 is in the form of an umbrella valve 850 positioned at the first opening 411 formed in the base plate 410, at the interface between the first fluid passageway 413 and the fluid chamber 480 of the example valve device 400. The example flow control device 800 may allow for flow in one direction and inhibit flow in a second direction.

FIG. 8A illustrates the example flow control device 800 in the example valve device 400, in the open position. FIG. 8B illustrates the example flow control device 800 in the example valve device 400, in the closed position. FIG. 8C is a perspective view of the example flow control device 800, in the form of the umbrella valve 850.

The example umbrella valve 850 includes a body portion 852, with a first flange portion 854 extending outward, for example radially outward, from a first end portion of the body portion 852, and a second flange portion 856 extending outward, for example, radially outward, from a second end portion of the body portion 852. At least one opening 858 is defined in the first flange portion 854.

In the arrangement shown in FIGS. 8A and 8B, the flow control device 800, in the form of the umbrella valve 850, floats in the first opening 411/first fluid passageway 413 of the base plate 410. The at least one opening 858 in the first flange portion 854 of the umbrella valve 850 is aligned with the first fluid passageway 413/first opening 411, to allow fluid to flow through the at least one opening 858 and into the first fluid passageway 413. For example, in the open state of the valve device 400 shown in FIG. 8A, a force of the fluid, flowing in the direction of the arrows F1 moves the umbrella valve 850 upward (in the example orientation shown in FIG. 8A), so that the first flange portion 854 is moved away from or spaced apart from the first opening 411. This allows fluid to flow through the at least one opening 858 in the first flange portion 854 into the first fluid passageway 413, through the fluid chamber 480 and into the second fluid passageway 414 in the direction of the arrows F1. Thus, the force of the fluid, flowing in the direction of the arrows F1, causes the umbrella valve 850 to move from an at rest position, in an upward direction, to the position shown in FIG. 8A, allowing fluid to flow into and through the valve device 400 in the direction of the arrows F1.

In the closed position of the valve device 400 shown in FIG. 8B, the diaphragm 420 (and the isolation layer 430/piezoelectric element 440 coupled thereto) is in an at rest, un-deflected, or un-deformed state, and the second flange portion 856 of the umbrella valve 850 is positioned so as to close the first opening 411 of the base plate 410. In the arrangement shown in FIG. 8B, the flow of fluid is thus blocked between first fluid passageway 413 and the fluid chamber 480.

In some examples, a spike in pressure, or force, or back pressure in the direction of the arrow F2 may cause the diaphragm 420 (and piezoelectric element 440/isolation layer 430) to deflect or deform, as shown in the enlarged portion of FIG. 8B. This deformation of the diaphragm 420 may allow fluid to flow from the second fluid passageway 414 into the fluid chamber 480 through the second opening 412. In the example shown in FIG. 8B, the first opening 411 remains covered, or blocked by the second flange portion 856 of the umbrella valve 850. This positioning of the second flange portion 856 across the first opening 411 prevents fluid from flowing from the fluid chamber 480 into the second fluid passageway 414 in response to the spike in pressure in the direction of the arrow F2, and unintentional opening of the valve device 400. Thus, the flow control device 800, in the form of the umbrella valve 850 positioned at the first opening 411 in the base plate 410, may maintain a closed state of the fluid chamber 480 and a corresponding closed state of the valve device 400, even in the event of a fluctuation or spike in pressure, or back pressure.

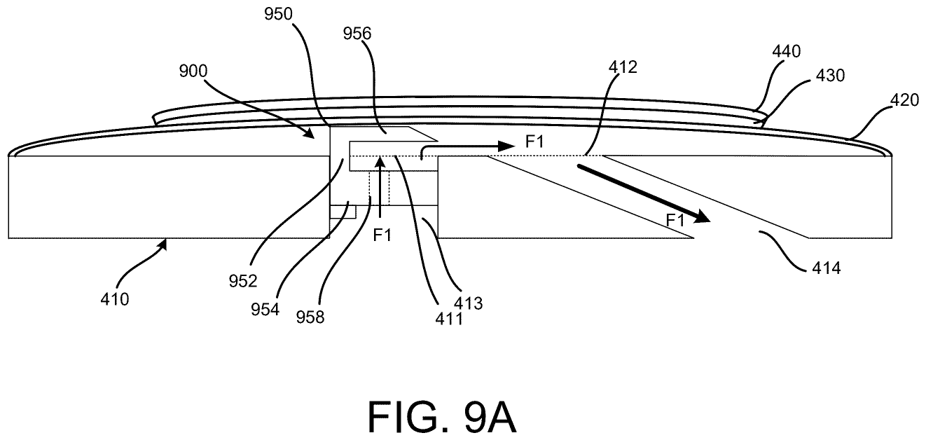

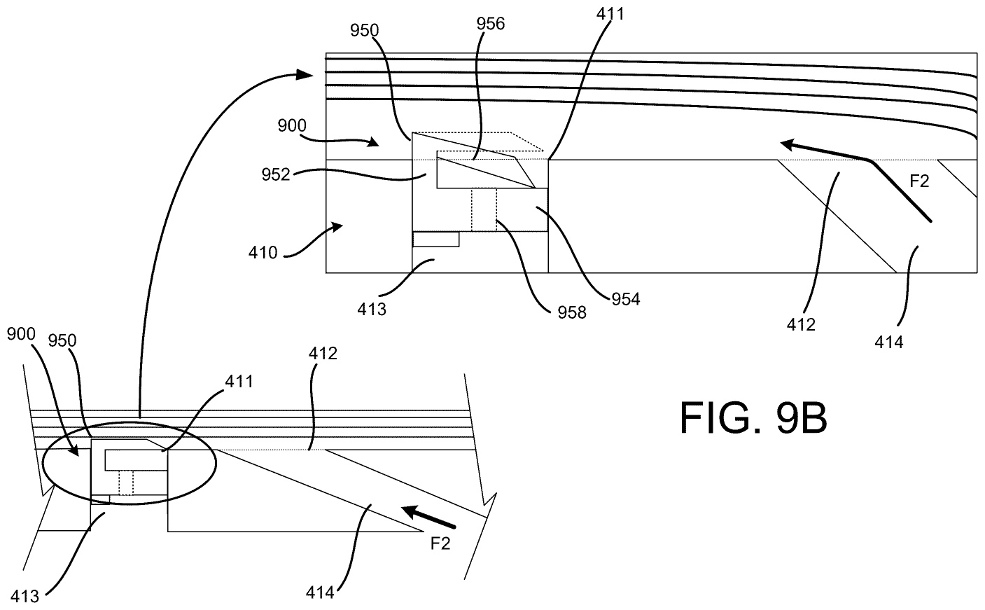

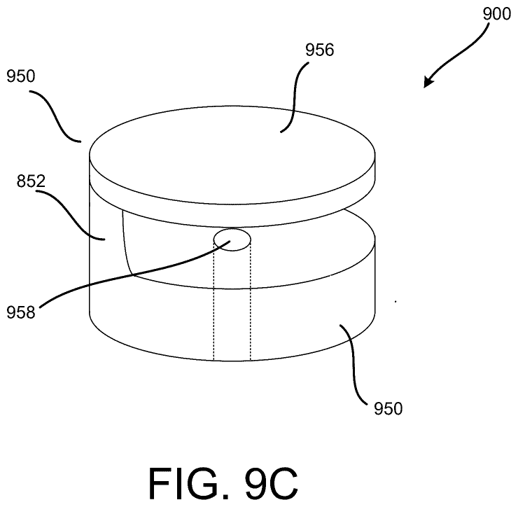

FIGS. 9A-9C illustrate an example auxiliary flow control device 900 that can be incorporated into the example valve device 400. In the example arrangement shown in FIGS. 9A-9C, the example auxiliary flow control device 900 is in the form of flap seal valve 950 positioned at the first opening 411 formed in the base plate 410, at the interface between the first fluid passageway 413 and the fluid chamber 480 of the example valve device 400. The example flow control device 900 may allow for flow in one direction and inhibit flow in a second direction.

FIG. 9A illustrates the example flow control device 900 in the example valve device 400, in the open position. FIG. 9B illustrates the example flow control device 900 in the example valve device 400, in the closed position.

The example flap seal valve 950 includes a body portion 952, with a base portion 954 a first end portion of the body portion 952, and a flap portion 956 at a second end portion of the body portion 952. At least one opening 958 is defined in base portion 954. In some examples, at least a portion of the flap seal valve 950 is made of an elastomer material, or a compliant material, which allows for some amount of flex or movement in response to the application of force or pressure. In some examples, at least the flap portion 956 of the flap seal valve 950 is made of an elastomer material, or a compliant material that allow for flexure of at least a portion of the flap seal valve 950. In the arrangement shown in FIGS. 9A and 9B, the flow control device 900, in the form of the flap seal valve 950, the base portion 954 may be fixed in the first fluid passageway 413, while the flap portion 956 remains movable.

In the open state of the valve device 400 shown in FIG. 9A, the flap portion 956 is separated from, or spaced apart from the base portion 954, leaving the at least one opening 958 open to the fluid chamber 480. In the open state of the valve device 400 shown in FIG. 9A, fluid from the first fluid passageway 413 flows into the fluid chamber 480 through the at least one opening 958, and out of the valve device 400 through the second fluid passageway 414, in the direction of the arrows F1.

In the closed position of the valve device 400 shown in FIG. 9B, the diaphragm 420 (and the isolation layer 430/piezoelectric element 440 coupled thereto) is in an at rest, un-deflected, or un-deformed state, and the flap portion 956 of the flap seal valve 950 is positioned so as to close the first opening 411 of the base plate 410. In the arrangement shown in FIG. 9B, the flow of fluid is thus blocked between first fluid passageway 413 and the fluid chamber 480.

In some examples, a spike in pressure, or force, or back pressure in the direction of the arrow F2 may cause the diaphragm 420 (and piezoelectric element 440/isolation layer 430) to deflect or deform, as shown in the enlarged portion of FIG. 9B. This deformation of the diaphragm 420 may allow fluid to flow from the second fluid passageway 414 into the fluid chamber 480 through the second opening 412. In response to this spike in pressure, or force, or back pressure and corresponding flow of fluid in the direction of the arrow F2, while the valve device 400 is in the closed position, at least a portion of the flap seal valve 950 may deflect, or deform, to maintain the sealed condition of the fluid chamber 480, and restrict flow through the fluid chamber 480 in the closed state of the valve device 400, as shown in the enlarged portion of FIG. 9B. In particular, in the example shown in FIG. 9B, the force of the fluid exerted on the flap seal valve 950 in the direction of the arrow F2 causes the flap portion 956 to deflect from an at rest position (shown in dashed lines) to a deflected position. This deflection of the flap portion 956 blocks the at least one opening 958 in the base portion 954, and thus blocks the flow of fluid from the fluid chamber 480 to the first fluid passageway 413. Thus, the flow control device 900, in the form of the flap seal valve 950 positioned at the first opening 411 in the base plate 410, may deform or deflect to maintain a closed state of the fluid chamber 480 and a corresponding closed state of the valve device 400, even in the event of a fluctuation or spike in pressure, or back pressure.

The architecture and principles of operation of the valve device described above also can be used to implement one or more pumps (such as pumps that pumps P1, P2, PV1, PV2 of FIGS. 3A and 3B) to pump fluid from one location to another. For example, repeated movement of a diaphragm between an open position and a closed position, relative to a base plate, can cause fluid to be drawn into a chamber formed between the diaphragm and the base plate through a first fluid passageway and expelled out of the chamber into a second fluid passageway. In this manner, fluid can be pumped from a first location that is fluidically connected to the first passageway to a second location that is fluidically connected to the second passageway. In some implementations, one or more one-way valves can be configured to prevent, or limit, the flow of fluid in the direction from the second location to the first location.

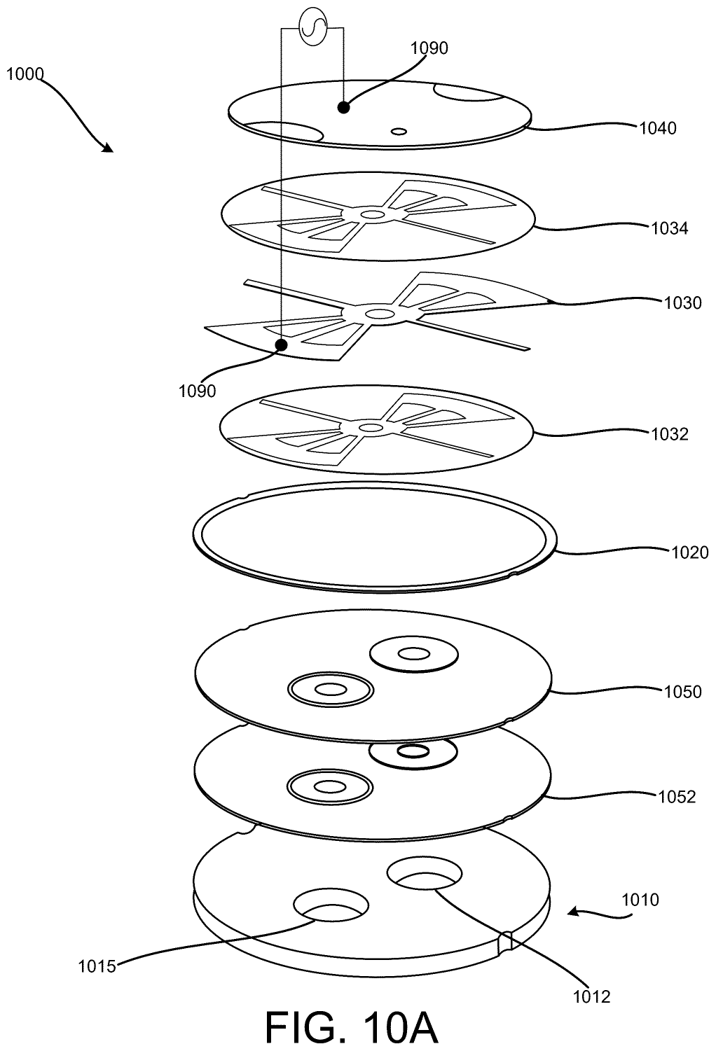

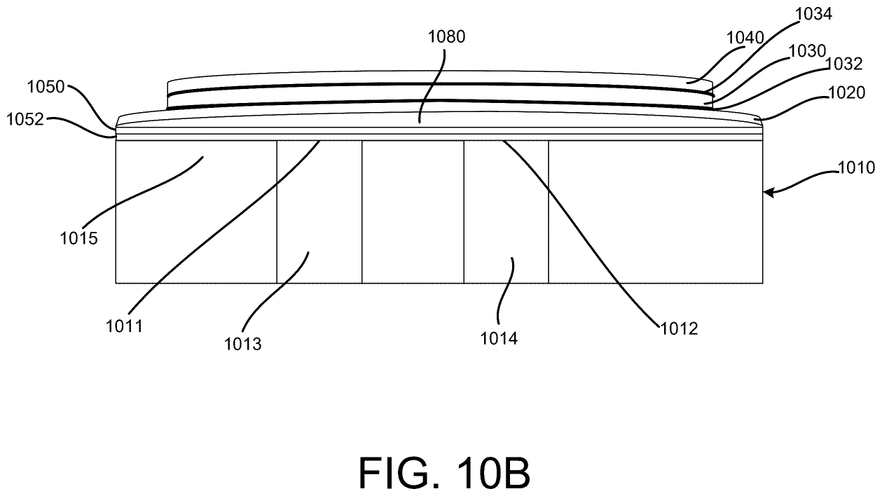

FIG. 10A is a partially exploded perspective view of an example pump device 1000, and FIG. 10B is a cross-sectional view of the example pump device 1000. The example pump device 1000 shown in FIGS. 10A-10B is an example of a fluid control device, or a fluidic component, included in the fluid control system 206 of the example electronically controlled fluid manifold 230 described above.

In the example arrangement shown in FIGS. 10A-10B, the example pump device 1000 includes a base plate 1010 defining a base portion of the pump device 1000. A diaphragm 1020 is positioned on the base plate 1010. A piezoelectric element 1040 is positioned on the diaphragm 1020, with an isolation layer 1030 positioned between the diaphragm 1020 and the piezoelectric element 1040. The piezoelectric element can be electrically powered (e.g., by a battery of the implantable fluid-operated inflatable device 100) to drive the diaphragm 1020 to pump fluid through the pump device 1000. The diaphragm 1020 can include a thin metal foil, whose shape can be repeatably deformed in response to movement by the piezoelectric element 1040. In some implementations, the diaphragm 1020 can include titanium material. In some implementations, the diaphragm 1020 can include gold material. In some implementations, the diaphragm 1020 can include stainless steel material or other alloys. In some implementations, the isolation layer 1030 can include a polyamide material that has a high resistivity, for example, a resistivity greater than 10¹³ Ohm-cm to provide electrical isolation between the piezoelectric element 1040 and the diaphragm 1020.

In some examples, an epoxy layer 1032 provides for the coupling of the isolation layer 1030 and the diaphragm 1020. In some examples, an epoxy layer 1034 provides for the coupling of the piezoelectric element 1040 and the isolation layer 1030, and the epoxy layers 1032, 1034 together provide for the coupling of the piezoelectric element 1040 to the diaphragm 1020. In some implementations, the epoxy layers 1032, 1034 are not distinct but are part of one epoxy layer. The epoxy layers 1032, 1034 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 1090 are arranged on the example pump device 1000. In the example shown in FIG. 10A, the example pump device 1000 includes a pair of electrodes 1090 coupled between the isolation layer 1030 and the piezoelectric element 1040. Application of a voltage to the piezoelectric element 1040 causes a deflection or deformation of the piezoelectric element 1040 and a corresponding deflection or deformation of the diaphragm 1020 coupled thereto.

When the pump device 1000 is used in the fluid control system 206 of the example electronically controlled fluid manifold 230 described above, the piezoelectric element 1040 can be controlled to cause fluid to be pumped by device 1000, for example, by repeatedly changing a volume of the fluid chamber 1080 by deforming the deformable diaphragm 1020 to pump fluid from the fluid reservoir to the inflatable member.

In the example arrangement shown in FIGS. 10A-10B, a fluid chamber 1080 is defined between the base plate 1010 and the diaphragm 1020. The base plate 1010 includes a first opening 1015 that provides for communication between a first fluid passageway 1013 and the fluid chamber 1080. The base plate 1010 includes a second opening 1012 that provides for communication between a second fluid passageway 1014 and the fluid chamber 1080. In some examples, the diaphragm 1020 can be actuated to move between a closed position in which the diaphragm 1020 is proximate to the base plate 1010 due to the deflection of the diaphragm 1020, such that the volume of the chamber 1080 is minimized, and an open position in which the base plate 1010 is separated, or spaced apart from, the diaphragm 1020 due to the deflection of the diaphragm 1020, such that the volume of the chamber is maximized. When the diaphragm 1020 is actuated to move from the closed position to the open position, fluid can be drawn into the chamber 1080 through the first fluid passageway 1013, and when the diaphragm 1020 is actuated to move from the open position to the closed position, fluid can be expelled from the chamber 1080 through the second fluid passageway 1014. Repeatedly actuating the diaphragm between the closed and open position allows fluid to be pumped through the pump device 1000, from the first fluid passageway 1013 to the second fluid passageway 1014 via the fluid chamber 1080.

In some implementations, the pump device 1000 can include one or more foil plates 1050 and 1052 to control the flow of fluid into and out of the pump device 1000. The foil plates 1050, 1052, as explained in more detail herein, can include one-way check valves that operate to permit fluid to flow in one direction through the values but not in an opposite direction. The one-way check valves defined by the one or more foil plates can be positioned in, or in fluid connection with, a fluid passageway 1013, 1014 of the pump device 100. In some examples, a check valve is positioned in, or in fluid connection with, a portion of a fluid passageway 1013, 1014 so as to inhibit the unintended flow of fluid through the pump device in the event of a fluctuation, or spike in pressure. In some examples, a check valve is positioned in a fluid passageway 1013, 1014 so as to counteract a back pressure that would otherwise overcome the closing pressure and cause unintentional flow through the pump device 1000. In some example implementations, a first check valve defined by one or more foil plates 1050, 1052 is positioned in, or in fluid connection with (e.g., at a first opening 1011 of), a first fluid passageway 1013 of the pump device and is configured to permit fluid to easily flow from the first fluid passageway 1013 into the chamber 1080 but to prevent or inhibit the flow of fluid from the chamber 1080 into the passageway 1013. In some example implementations, a second check valve defined by one or more foil plates 1050, 1052 is positioned in, or in fluid connection with (e.g., at a first opening 1012 of), a second fluid passageway 1014 of the pump device 1000 and is configured to permit fluid to easily flow from the chamber 1080 into the second fluid passageway 1013 but to prevent or inhibit the flow of fluid from the passageway 1013 into the chamber 1080.

Application of an alternating current (AC) voltage to the piezoelectric element 1040 can cause the diaphragm 1020 of the pump device 1000 to oscillate between a first position that defines the closed position of the chamber 1080, in which the diaphragm 1020 is proximate to the base plate 1010 and the volume of the chamber 1080 is minimized, and a second (e.g., domed) position that defines the open position of the chamber 1080, in which the diaphragm 1020 is separated from the base plate and the volume of the chamber 1080 is maximized. As the diaphragm 1020 of the pump device 1000 oscillates between a first position and the second position, fluid is drawn into the chamber 1080 from the first passageway 1013 and is expelled from the chamber 1080 into the second passageway 1014. As the diaphragm 1020 of the pump device 1000 oscillates between a first position and the second position, the one-way check valves defined by the one or more foil plates 1050, 1052 prevent or inhibit fluid from flowing from the chamber 1080 into the first passageway 1013 and prevent or inhibit fluid from flowing into the chamber 1080 from the second passageway 1014. Thus, the application of the AC voltage to the piezoelectric element 1040 causes the pump device 1000 to pump fluid from the first passageway 1013 to the second passageway 1014.

The frequency of the AC voltage applied to the piezoelectric element 1040 can determine an oscillation mode of the piezoelectric element 1040. In some implementations, the frequency of the AC voltage is selected to excite a lowest-order mode in which the center of the circular piezoelectric element 1040 experiences the greatest extent of movement during an oscillation cycle, such that an amount of fluid pumped during an oscillation cycle is maximized compared to other oscillation modes.

The piezoelectric element 1040 can be controlled to cause fluid to be pumped by device 1000, for example, by repeatedly changing a volume of the fluid chamber 1080 by deforming the deformable diaphragm 1020 to pump fluid from the fluid reservoir to the inflatable member.

The volume of the chamber 1080 can be determined, at least in part, by the shape, geometry, and material properties of the components used to form the chamber 1080, including, for example, the base plate 1010 and the deformable diaphragm 1020. In some cases, a relatively larger volume of the chamber 1080, for an approximately constant diameter of the chamber, can result in more fluid being pumped in each open/close cycle of the pump 1000. To achieve a relatively larger volume of chamber 1080, the deformable diaphragm can be deformed or biased into a non-flat dome-shaped configuration before it is attached to the piezoelectric element 1040.

As explained above in connection with FIG. 4H, in some implementations, before the diaphragm 1020 is placed in attached to the piezoelectric element 1040, a voltage can be placed across the electrodes 1090 attached to the piezoelectric element 1040 to configure the piezoelectric element 1040 in the domed configuration that is assumes when the fluid chamber is in the open position (See FIG. 4D). Then, the diaphragm can be placed in contact with the piezoelectric element while the piezoelectric element 440 is in its domed configuration, and the epoxy can be cured when the piezoelectric element and the diaphragm 420 are in the domed configuration, which can reduce stress on the adhesive bond between the diaphragm 420 and the piezoelectric element 440.

However, when the diaphragm 1020 is naturally unstressed when it is in a flat configuration and then is attached to the piezoelectric element 1040 that is the domed configuration, the diaphragm 1020 then can be in a stressed position, such that internal stresses within the diaphragm generate a force that oppose the forces that place the piezoelectric element 1040 in the dome configuration. Because of this, the height of the dome can be somewhat less than would be the case if the diaphragm 1020 were unstressed when it was attached to the piezoelectric element 1040 when the element is in its domed configuration.

Therefore, to accomplish a relatively higher dome height and chamber volume, the diaphragm 1020 can be configured into a non-flat (e.g., dome-shaped) configuration before it is attached to the piezo electric element 1040. Different techniques can be used to accomplish the non-flat configuration of the diaphragm 1020, including, for example, mechanical and/or thermal techniques.

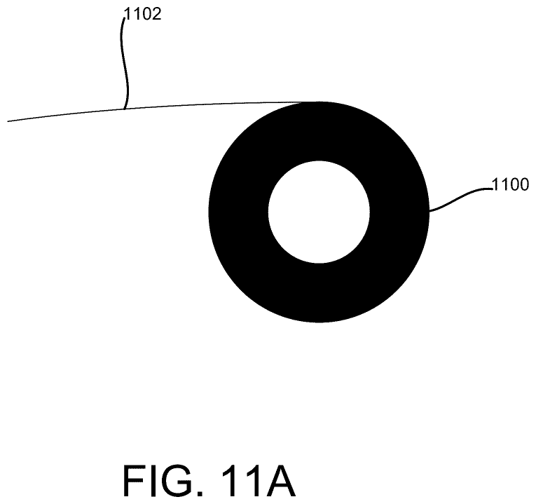

For example, in one implementation, the diaphragm 1020 can be fabricated from a sheet of metal material that is not inherently flat in its under stressed condition, but rather that has an inherent curvature. FIG. 11A is a schematic diagram of a roll 1100 of metal foil that could have been fabricated from a, for example, roll-milling process that causes a natural curvature in the foil of the roll. Therefore, when a sheet of material 1102 is taken from the roll 1100, the sheet of material 1102 will have a natural curvature in its unstressed condition.

FIG. 11B is a schematic diagram of a non-flat, unstressed diaphragm 1020 attached to a base plate 1010. The non-flat diaphragm 1020 can be placed on a top surface 1104 (which may be flat) of the base plate 1010, and then when the diaphragm is correctly positioned it can be attached to the base plate. In some implementations, the diaphragm 1020 can be welded at welding joints 1110 around a perimeter of the diaphragm to the base plate 1010. The welding of the joints 1110 can be accomplished through, for example, TIG welding or laser welding. Once the diaphragm 1020 is attached to the base plate 1010, the piezoelectric element 1040 can be attached to the diagram 1020.

When the diaphragm 1020 is formed from a sheet of material 1102 that is not flat in its unstressed state, the diaphragm 1020 can have a natural curvature in its unstressed state that can approximately match the curvature of the piezoelectric element 1040 when the piezoelectric element is in its dome-shaped configuration. Because of this, relatively little force is transmitted from the diaphragm 1020 to the piezoelectric element 1040 when the diaphragm is attached to the piezoelectric element and the piezoelectric element is in its dome-shaped configuration, so that a higher dome height and chamber volume can be achieved.

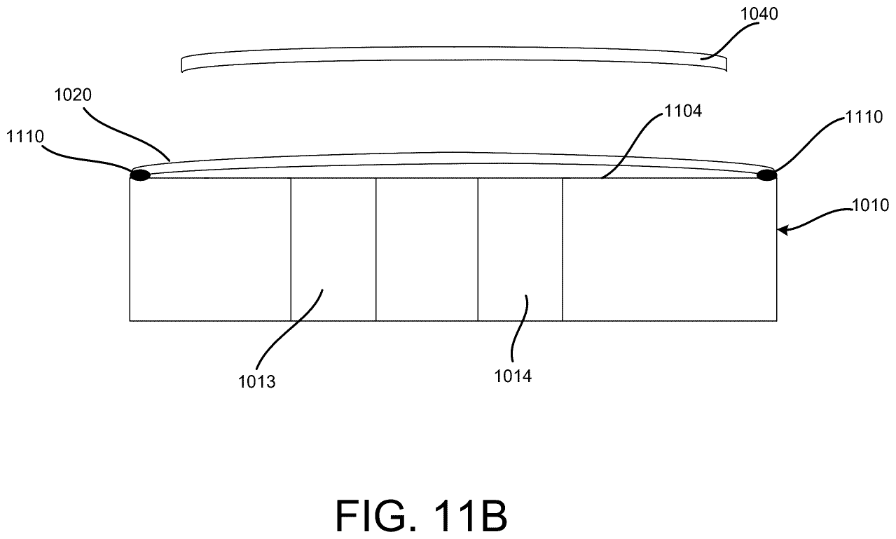

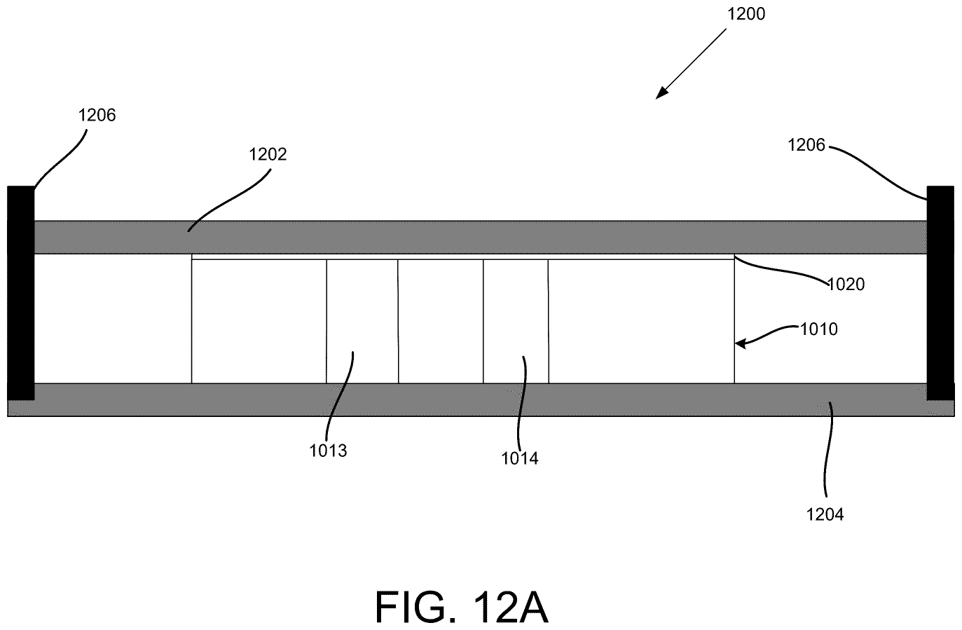

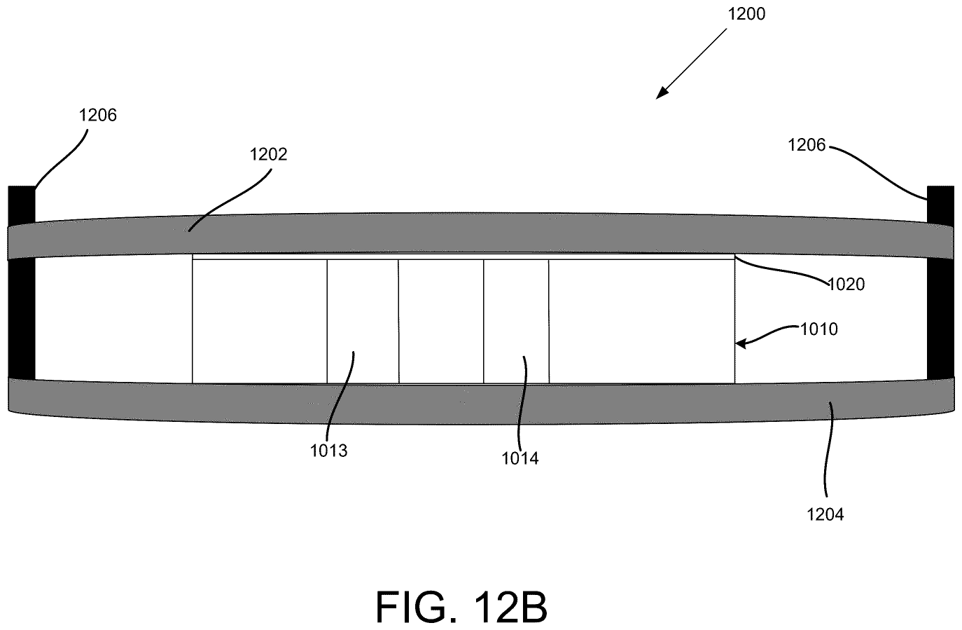

FIGS. 12A and 12B are schematic cross-sectional diagrams of a system 1200 for creating a non-flat diaphragm in an unstressed state that can be used in the fluidic pumps described herein. The system 1200 can include a fixture top plate 1202, a fixture bottom plate 1204, and a plurality of bolts 1206 that couple the top plate 1202 to the bottom plate 1204. The top plate 1202 and the bottom plate 1204 can be generally circular in shape, and the plurality of bolts 1206 can be located generally at the perimeters of the plates 1202, 1204. The generally circular base plate 1010 and the generally circular diaphragm 1020 located on top of the base plate can be sandwiched between the top plate 1202 and the bottom plate 1204, with the bolts 1206 located on a pitch circle having a diameter greater than the diameter of the base plate 1010.

The bolts 1206 can be used to adjust a force with which the top plate 1202 and the bottom plate 1204 are pulled towards each other. In FIG. 12A, the bolts 1206 are adjusted to provide a relatively low force, such that the plates 1202, 1204 are generally flat, and in FIG. 12B, the bolts 1206 are adjusted to provide a relatively high force that bends the plates 1202, 1204 into a domed configuration, in which the centers of the plates are farther away from each other than the perimeters of the plates. When the base plate 1010 and the diaphragm 1020 are sandwiched between the top plate 1202 and the bottom plate 1204 and the bolts 1206 are tightened between the plates 1202, 1204, the higher force on the perimeter of the diaphragm 1020, relative to the center of the diaphragm, causes the diaphragm to also take on a domed configuration. This can cause a plastic deformation of the diaphragm 1020, such that the diaphragm 1020 takes on a permanent slightly domed shape, although the base plate may not be plastically deformed due to its greater thickness as compared to that of the diaphragm 1020.

After deforming the diaphragm 1020, the bolts can be unscrewed from the plates 1202, 1204, and then with the diaphragm 1020 in its domed configuration and the base plate in its original flat configuration, the diaphragm 1020 can be welded at the perimeter of the diaphragm to the base plate 1010. The diaphragm 1020 welded to the base plate can be removed from the system 1200 for use in a fluidic pump. By welding the diaphragm 1020 to the base plate 1010 when the diaphragm is in its domed configuration, the diaphragm 1020 can have a natural curvature in its unstressed state that its shape can approximately match the curvature of the piezoelectric element 1040 when the piezoelectric element is in its dome-shaped configuration. Because of this, relatively little force is transmitted from the diaphragm 1020 to the piezoelectric element 1040 when the diaphragm is attached to the piezoelectric element, when the piezoelectric element is in its dome-shaped configuration, so that a higher dome height and chamber volume can be achieved.

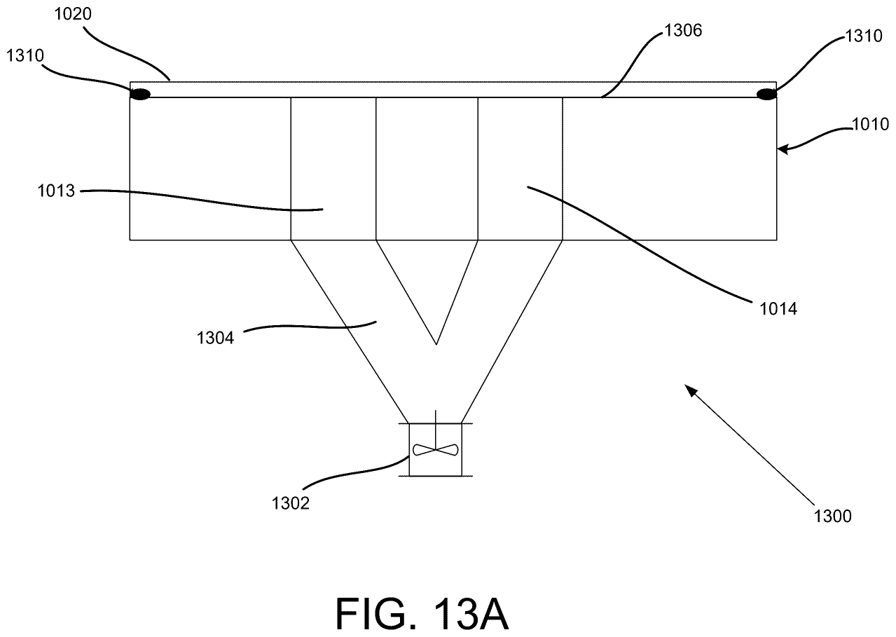

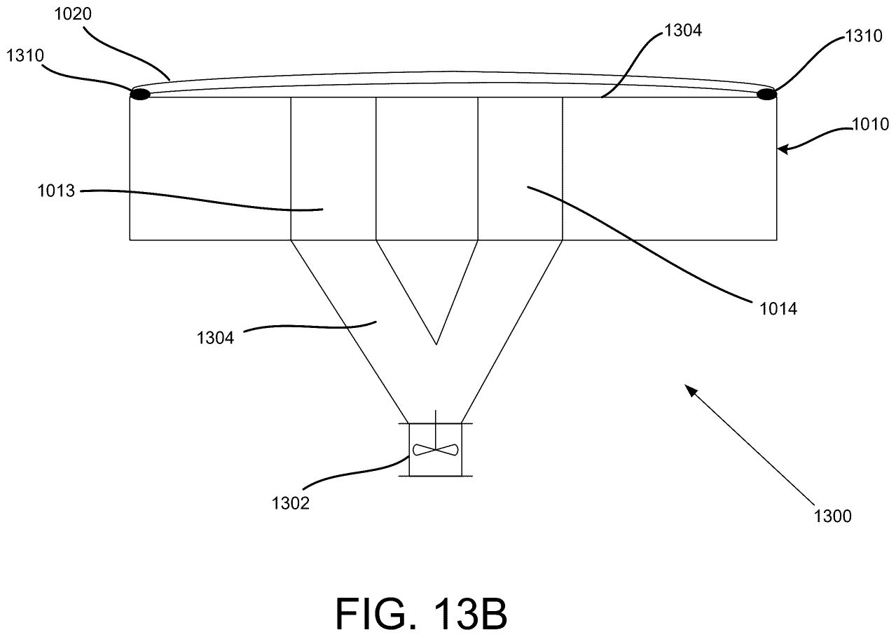

FIGS. 13A and 13B are schematic cross-sectional diagrams of another system 1300 for creating a non-flat diaphragm in an unstressed state that can be used in the fluidic pumps described herein. The system 1300 can include a fluid (e.g., liquid or gas) pump 1302 and one or more conduits 1304 between the fluid pump and the base plate 1010. The fluid pump 1302 can direct fluid (e.g., liquid or gas) through the one or more conduits 1304 and into the passageways 1013, 1014 from the bottom side of the base plate.

As shown in FIG. 13A, the diaphragm 1020, in a flat configuration, can be positioned on a top surface 1306 of the base plate 1010, and then portions of the diaphragm can be spot welded to the base plate 1010 in a plurality of locations 1310 around a (e.g., circular) perimeter of the diaphragm. The portions of the diaphragm 1020 that are spot welded to the base plate 1010 can encompass less than 25%, less than 10%, less than 3%, or less than 1% of the perimeter of the diaphragm 1020.

Then, as shown in FIG. 13B, with the diaphragm 1020 positioned on a top surface 1306 of the base plate 1010, after the diaphragm 1020 is spot welded to the base plate 1010, the fluid pump 1302 can be activated to cause the flow of fluid through the passageways 1013, 1014 to impart a force to a central portion of the bottom side of the diaphragm 1020 where the outlets of the passageways are located. This force can cause the center of the diaphragm 1020 to move away from the top surface 1306 of the base plate. While the fluid pump 1302 forces fluid through the passageways 1013, 1014 to force the center of the diaphragm 1020 away from the top surface 1306 of the base plate, the rest of the perimeter of the diaphragm 1020 can be welded to the base plate 1010 to create a chamber between the base plate and the diaphragm. In this manner, the diaphragm 1020 can be attached to the base plate 1010 in a non-flat configuration before the piezoelectric element is attached to the diaphragm.

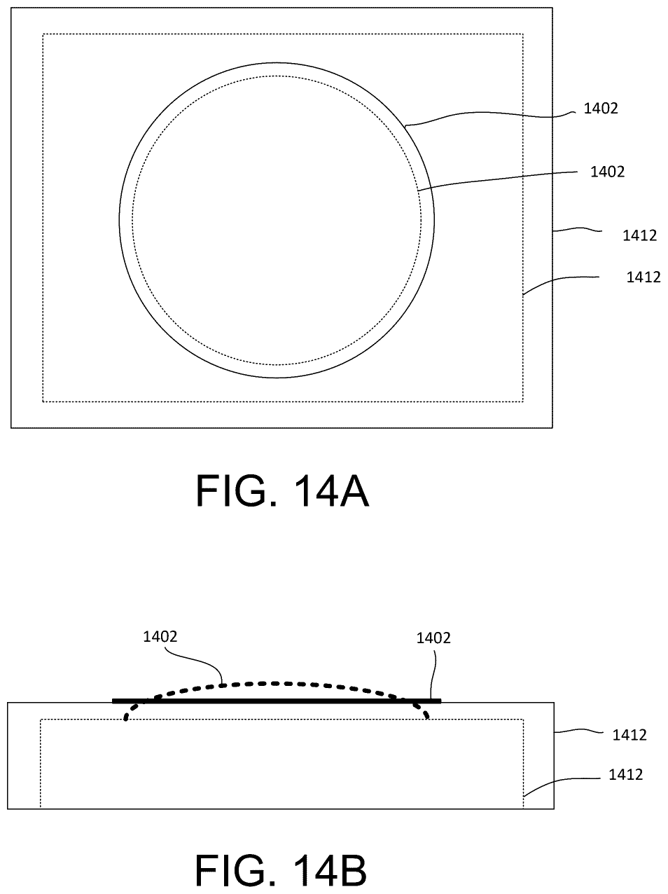

FIGS. 14A and 14B are top and side schematic views, respectively, of a metal diaphragm 1402 being attached a metal base plate 1412, where the thermal expansion of the metal materials is used to create a dome-shaped diaphragm on the base plate. When the base plate 1412 has a first temperature (e.g., room temperature or body temperature) it can have smaller dimensions, shown in dotted lines, than the dimensions, shown in solid lines, when the base plate is at a second temperature (e.g., 400° C.) that is greater than the first temperature. The diaphragm 1402, when in a flat configuration (shown in solid lines), can be placed on the surface of the base plate 1412 when the base plate is at the second temperature and the temperature of the diaphragm 1402 is lower than the second temperature. Then, the perimeter of the diaphragm 1402 can be welded to the base plate before the diaphragm 1402 and the base plate reach thermal equilibrium. When the base plate cools to the first temperature and its dimensions shrink due to thermal contraction, the diameter of the weld joints between the diaphragm 1402 and the base plate can decrease, causing the center of the diaphragm to move away from the top surface of the base plate 1412 and to take on a domed configuration. In this manner, the diaphragm 1402 can be attached to the base plate 1412, such that the diaphragm has a non-flat configuration when the piezoelectric element is attached to the diaphragm.

The temperature of the base plate 1412 can be raised above the temperature of the diaphragm 1402 in a variety of different ways before the diaphragm 1402 is welded to the base plate 1412. For example, the base plate 1412 can be heated in an oven, or the base plate 1412 can be heated in a bath of hot liquid (e.g., boiling water), or the base plate (or portions thereof) can be heated by a laser, etc. In some implementations, when the diaphragm 1402 is laser welded to the base plate 1412, the base plate can be pre-heated with the laser that forms the weld joint before the diaphragm is welded to the base plate.

Referring again to FIG. 2, although considerable effort is expended to maintain the cleanliness of the components of the system and the purity of the fluid used within the system, it is still possible that some small amounts of foreign matter contaminate the fluid within the system. For example, when the reservoir 202, the inflatable members 204, and the housing 210 are implanted and connected (e.g., by conduits 203, 207) within a patient, it is possible that some contamination enters the fluidic system. In addition, it is possible that, once implanted within a patient, that small amounts of material disintegrate from walls of the reservoir 202, inflatable member 204, housing 210 and conduits 203, 207 and become suspended within fluid that flows within the system 200. Because of the small internal dimensions of the pumps and valves used within the fluidic system, the existence of particles of foreign matter suspended within the fluid flowing within the system poses a risk of clogging or damaging one or more of the pumps and valves, which may lead to malfunction of the system 200. To mitigate the effect of any particulate matter suspended within the fluid that flows within the system 200, the fluidic path can include one or more filters that block, or reduce the amount of, particulate matter that enters the pumps and valves of the system. In some implementations, the filters can be included in a fluid pathway of a pump or valve.

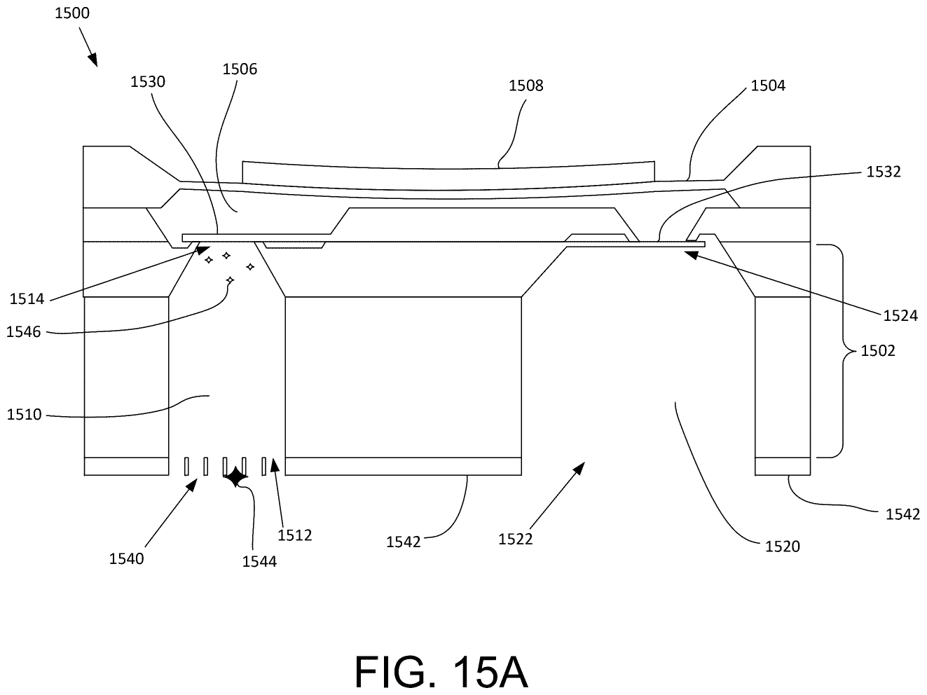

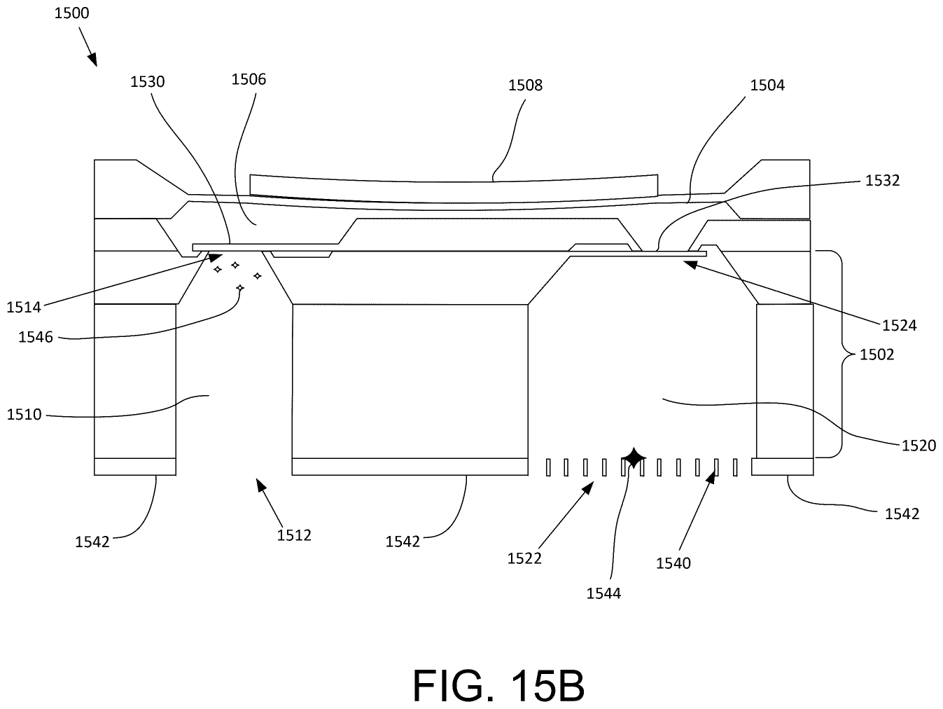

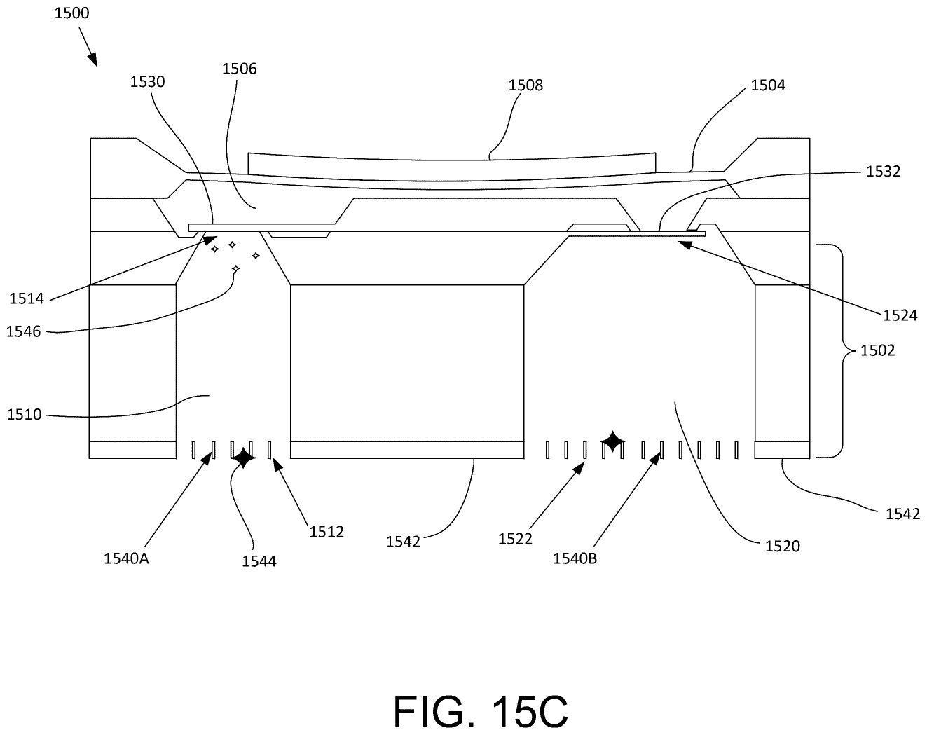

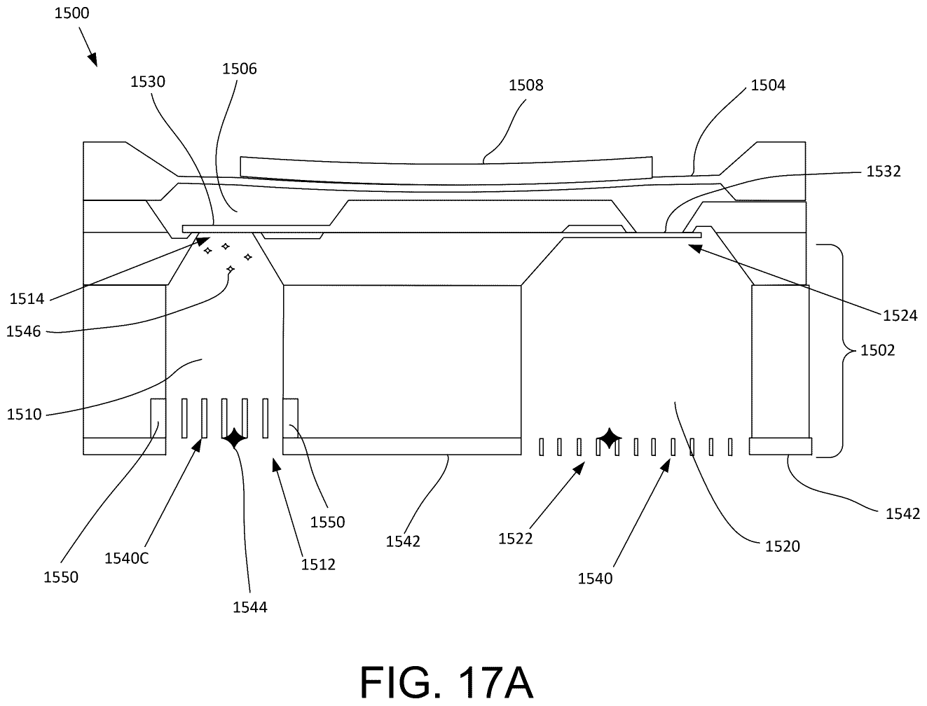

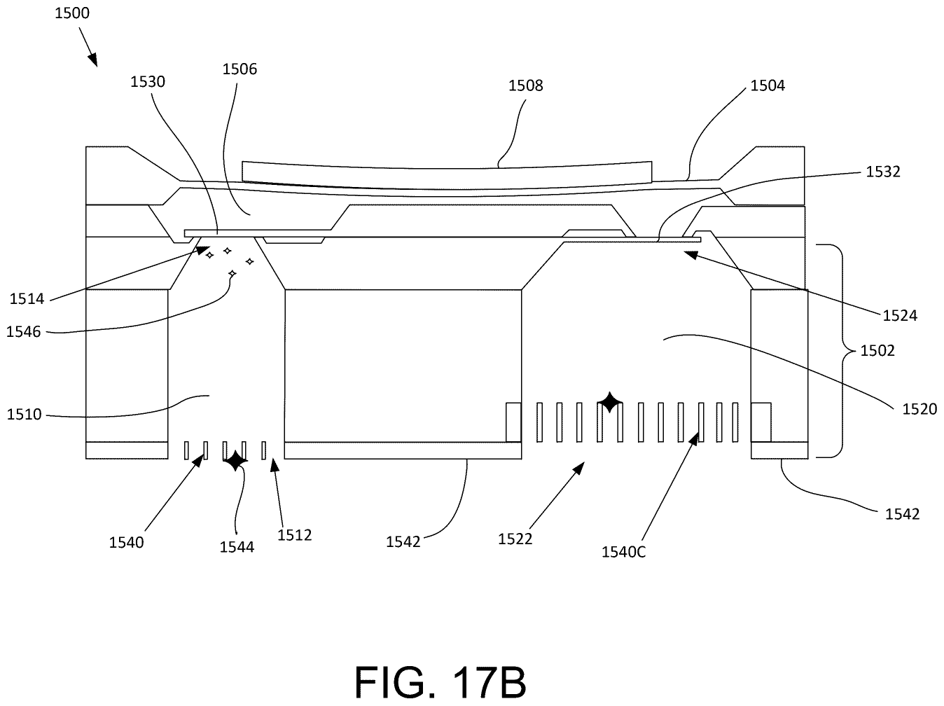

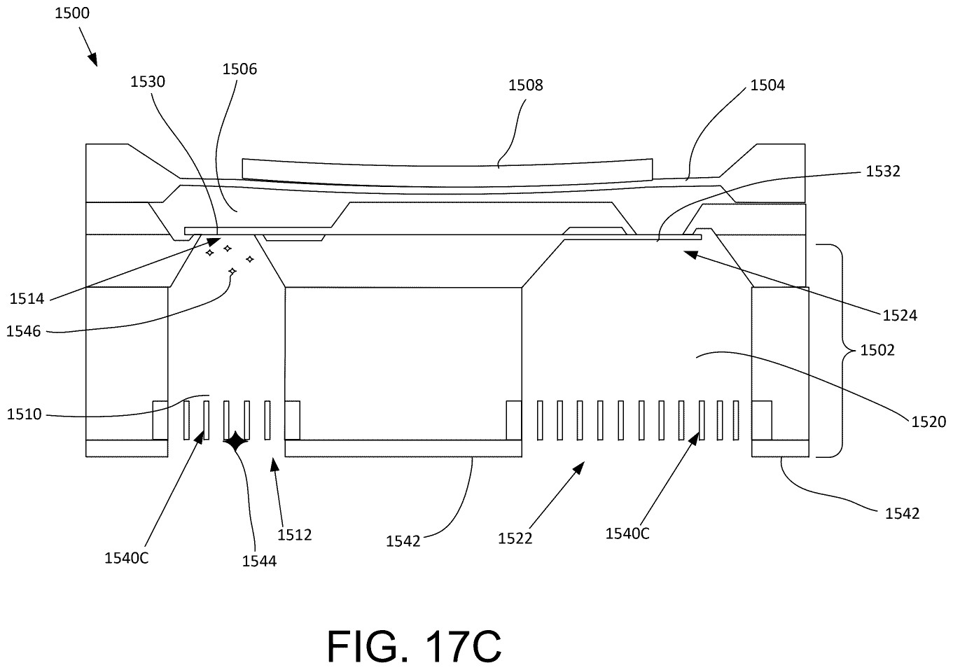

FIGS. 15A, 15B, 15C, 17A, 17B, and 17C are cross-sectional views of example pump devices 1500 that includes a filter for capturing particulate matter in the fluid flow and/or for blocking the particulate matter from entering certain parts of the fluidic system (e.g., for blocking particulate matter from entering a pump chamber of the device). The example pump device 1500 shown in FIGS. 15A, 15B, 15C, 17A, 17B, and 17C are examples of a fluid control device, or a fluidic component, included in the fluid control system 206 of the example electronically controlled fluid manifold 230 described above.

In the example arrangements shown in FIGS. 15A, 15B, 15C, 17A, 17B, and 17C, the example pump device 1500 includes a base plate 1502 defining a base portion of the pump device 1500. A diaphragm 1504 is positioned above the base plate 1502, and a fluid chamber 1506 is defined between the base plate 1502 and the diaphragm 1504. A piezoelectric element 1508 is positioned on the diaphragm 1504. The piezoelectric element can be electrically powered (e.g., by a battery of the implantable fluid-operated inflatable device) to drive the diaphragm 1504 to pump fluid through the pump device 1500. The diaphragm 1504 can include a thin metal foil, whose shape can be repeatably deformed in response to movement by the piezoelectric element 1508. In some implementations, the diaphragm 1504 can include titanium material.

The base plate 1502 can define a first fluid passageway 1510 through which fluid can flow from a fluid reservoir into the fluid chamber 1506. The first fluid passageway 1510 can include an opening 1512 at a first end of the passageway 1510, which is distal to the fluid chamber 1506, and can include an opening 1514 and a second end of the passageway 1510, which is proximate to the fluid chamber 1506. The base plate 1502 can define a second fluid passageway 1520 through which fluid can flow from the fluid chamber 1506 two and inflatable member. The second fluid passageway 1520 can include an opening 1522 at a first end of the passageway 1520, which is distal to the fluid chamber 1506, and can include an opening 1524 and a second end of the passageway 1520, which is proximate to the fluid chamber 1506. In some implementations, the first fluid passageway 1510 and the second fluid passageway 1520 can be tapered, such the passageways 1510, 1520 have larger cross-sectional areas at the ends 1512, 1522 of the passageways that are distal to the fluid chamber 1506 than at ends of the passageways that are proximate to the fluid chamber.

The pump device 1500 can include a first flexible flap 1530 that includes a portion that having an area that is greater than an area of the passageway opening 1514 that is proximate to the fluid chamber 1506 and that covers the opening, such that the first flexible flap 1530 is configured to seal against portions of the base plate that defines the opening 1514 of the first fluid passageway 1510 to close the opening 1514 when a fluid pressure in the fluid chamber 1506 is greater than a fluid pressure of fluid in the first fluid passageway 1510. The flexible flap 1530 can be secured to the base plate over a portion of its extent but can have a portion that is unsecured, such that at least a portion of the flexible flap is configured to be pushed away from one or more walls of the fluid passageway 1510 that defines the opening 1514 when a fluid pressure of fluid in the first fluid passageway 1510 is greater than a fluid pressure in the fluid chamber 1506. In this manner, the flexible flap 1530 operates to allow fluid to flow from the first fluid passageway 1510 into the fluid chamber 1506 but to block the flow of fluid from the fluid chamber 1506 into the first fluid passageway 1510. The flexible flap 1530 can be made of a variety of materials including, for example, titanium, elastomeric material, plastic material, etc.

The pump device 1500 can include a second flexible flap 1532 that includes a portion that having an area that is greater than an area of the passageway opening 1524 that is proximate to the fluid chamber 1506 and that covers the opening, such that the second flexible flap 1532 is configured to seal against portions of the base plate that defines the opening 1524 of the second fluid passageway 1520 to close the opening 1524 when a fluid pressure in the fluid chamber 1506 is greater than a fluid pressure of fluid in the second fluid passageway 1520. The flexible flap 1532 can be secured to the base plate over a portion of its extent but can have a portion that is unsecured, such that at least a portion of the flexible flap is configured to be pushed away from one or more walls of the second fluid passageway 1520 that defines the opening 1524 when a fluid pressure in the fluid chamber 1506 is greater than a fluid pressure of fluid in the second fluid passageway 1520. In this manner, the flexible flap 1532 operates to allow fluid to flow from the fluid chamber 1506 into the second fluid passageway 1520 but to block the flow of fluid from the second fluid passageway 1520 into the fluid chamber 1506. The flexible flap 1532 can be made of a variety of materials including, for example, titanium, elastomeric material, plastic material, etc.

With the flexible flaps 1530, 1532 configured in this way to allow fluid to flow in a first direction from the first fluid passageway 1510 into the fluid chamber 1506 and out of the fluid chamber into the second fluid passageway 1520 but not in a direction opposite to the first direction, repeated expansion and contraction of the volume of the fluid chamber 1506 in response to the piezoelectric element 1508 operating on the deformable diaphragm 1504 can cause fluid to be pumped from a reservoir fluidically connected to the first fluid passageway 1510 to an inflatable member that is fluidically connected to the second fluid passageway 1520.

The pump device 1500 can include a fluid filter 1540 that is located within, or at the end 1512 of, the first fluid passageway 1510 or that is located within, or at the end 1522 of, the second fluid passageway 1520. The fluid filter 1540 can operate to block, for example, debris, foreign matter, particulates suspended in the fluid flowing through the device 1500 from passing through the first fluid passageway 1510 and into the fluid chamber 1506 and/or from exiting the second fluid passageway 1520. For example, as shown in FIG. 15A, a fluid filter 1540 is located at the opening 1512 into the first fluid passageway 1510. As shown in FIG. 15B, a fluid filter 1540 is located at the opening 1522 into the second fluid passageway 1520. As shown in FIG. 15C, a fluid filter 1540A is located at the opening 1512 into the first fluid passageway 1510, and a fluid filter 1540B is located at the opening 1522 into the second fluid passageway 1520.

In some implementations, the fluid filter 1540, 1540A, 1540B can include a metal foil (e.g., a titanium foil, having a pattern of openings that permit fluid to flow through the openings but that block particulates having a characteristic size larger than a threshold size from flowing through the opening. For example, particulates 1544 having a characteristic size (e.g., minimum transverse extent) that is greater than a threshold size defined by the size (e.g., diameter) of the openings can be blocked by the filter 1540, while particulates 1546 and a characteristic size smaller than the threshold size can pass through the filter 1540.

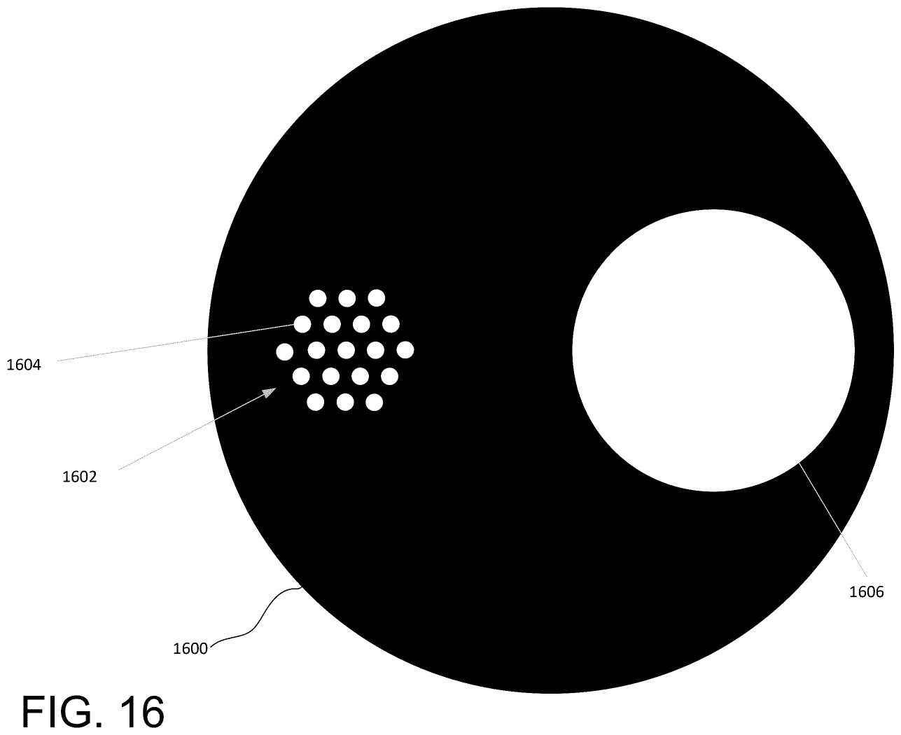

FIG. 16 is a schematic end view of a filter foil 1600. In some implementations, the filter foil 1600 can be made of metal (e.g., titanium) and can have a first section 1602 that includes a plurality of openings 1604. The openings can have a variety of different shapes, including circular, oblong, square, rectangular, hexagonal, etc. The plurality of openings 1604 can be arranged in a regular or irregular pattern. For example, the openings 1604 can be arranged in a two-dimensional hexagonal pattern, as shown in FIG. 16, or in a square pattern, or another type of regular or irregular pattern.

The plurality of openings 1604 can be formed in the filter foil 1600 in a number of different ways. For example, in some implementations, the pattern of openings can be mechanically stamped into the metal foil 1600. In some implementations, the pattern of openings 1604 can be laser etched into the metal foil 1600. In some implementations, the pattern of openings can be chemically etched (e.g., through a lithographic process) into the metal foil 1600.

Referring again to FIG. 15A and also to FIG. 16, the section 1602 that includes the plurality of openings 1604 can be arranged on the filter foil 1600 so that the pattern of openings 1604 is aligned with the opening 1512 of the first fluid passageway 1510 when the filter foil 1600 is attached to the base plate 1502. The filter foil 1600 also can include an opening 1606 in the filter foil that is aligned with the opening 1522 of the second fluid passageway 1520 of the base plate 1502 when the filter foil is attached to the base plate.

In some implementations, the filter foil 1600 can be welded to the base plate 1502. For example, when the base plate includes titanium and the filter foil 1600 includes titanium, the filter foil 1600 can be welded to the titanium baseplate 1502. Prior to attempting (e.g., welding) the filter foil 1600 to the base plate 1502, the filter foil 1600 can be positioned relative to the openings 1512, 1520 in the baseplate, such that the first section 1602 of the filter foil, which includes the plurality of openings 1604, is positioned at the end of the first fluid passageway 1510 and such that the opening 1606 in the filter foil 1600 is positioned at the end of the second fluid passageway 1520. Similarly, when a filter foil is attached to the baseplate shown in FIG. 15B, a section of the filter foil having a plurality of openings can be aligned with the end of the second fluid passageway 1520, and a larger opening in the filter foil 1600 in the aligned with the end of the first fluid passageway 1510. Similarly, when a filter foil is attached to the baseplate shown in FIG. 15C, a first section having a plurality of openings can be aligned with the end of the second fluid passageway 1520 and a second section having a plurality of openings can be aligned with the end of the first fluid passageway 1510.

In implementations in which the first fluid passageway 1510 and the second fluid passageway 1520 are tapered, such the passageways 1510, 1520 have larger cross-sectional areas at the ends 1512, 1522 of the passageways that are distal to the fluid chamber 1506 than at ends of the passageways that are proximate to the fluid chamber, filters 1540, 1540A, 1540B positioned at the distal ends of the fluid passageways 1510, 1520 can have cross-sectional areas that are greater than the cross-sectional areas of the openings 1514, 1524 between the passageways 1510, 1520 and the fluid chamber 1506. Because of this the area of the filter that is active for trapping particulate matter can be larger than the areas of the openings 1514, 1524 between the passageways 1510, 1520 and the fluid chamber 1506. In some implementations the flow of fluid through the filter 1540, 1540A, 1540B can be reversed to dislodge some of the particulate matter that has been trapped by the filters from the filters.

For example, referring again to FIG. 3A, a fluid conduit C1 can be provided between a downstream side of valve V2 and a pump P1. When the pump P1 is configured similarly to the pump shown in FIG. 15A, with a filter 1540 at the end of the fluid passageway 1510, the fluid conduit C1 can be connect to the first fluid passageway 1510, so that when fluid is pumped from inflatable member(s) 204 to the reservoir 202 some of that fluid is pumped into the fluid passageway 1510 of pump P1. Then, with valve V1 closed, the fluid that enters the first fluid passageway 1510 of pump P1 can flow out of the distal end 1512 of the first fluid passageway 1510 and back to the reservoir 202. The fluid that flows out of the distal end 1512 of the first fluid passageway 1510 can flush debris and particulate matter out of the filter 1540. In some implementations, the conduit C1 can include a one-way valve that allow fluid to pass from valve V2 to pump P1 but not in the opposite direction. Other such fluid connections, for example, conduit C2 of FIG. 3A, can be used to flush debris and particulate matter out of filters used in the fluid control system.

The example pump devices 1500 shown in FIGS. 17A, 17B, 17C include filters 1540, 1540C for blocking particulate matter in the fluid from entering a pump chamber of the device or for circulating in the fluidic system in which the pump devices operate. The filter 1540 shown in FIG. 17A is disposed at the distal end 1512 of the first fluid passageway 1510, and the filter 1540 shown in FIG. 17B is disposed at the distal end 1522 of the second fluid passageway 1520. These filters 1540 can be similar to the filters 1540, 1540B, 1540B shown in FIGS. 15A-15C, in that the filters 1540 can include a plurality of openings in a foil, where the size of the openings is selected to block the passage of particles having a characteristic size greater than a threshold size and to allow fluid and particles having a characteristic size less than the threshold size to pass through the openings.

In some implementations, the example pump devices 1500 shown in FIGS. 17A, 17B, 17C can include filters 1540C disposed within the first fluid passageway 1510 or within the second fluid passageway 1520, for example, between the first end 1512 of the first fluid passageway 1510 and the opening 1514 at the second end of the first fluid passageway 1510 and/or between the first end 1522 of the second fluid passageway 1520 and the opening 1524 at the second end of the second fluid passageway 1520. For example, as shown in FIG. 17A, the example pump device 1500 can include a filter 1540C disposed within the first fluid passageway 1510. In another example, as shown in FIG. 17 B, the example pump device 1500 can include a filter 1540C disposed within the second fluid passageway 1520. In another example, as shown in FIG. 17C, the example pump device 1500 can include a filter 1540C disposed within the first fluid passageway 1510 and another filter 1540C disposed within the second fluid passageway 1520.

Referring to FIG. 17A, the filter 1540C can include an outer frame 1550 that supports material within the frame that includes a plurality of small openings or passages through which fluid can pass but which have a threshold size that blocks particles having a characteristic size greater than the threshold size from passing through the filter 1540C.

The outer frame 1550 can be secured to the base plate 1502 that defines the first fluid passageway 1510. In some implementations, the base plate 1502 can define a receptacle that receives the outer frame 1550. In some implementations, the receptacle can have a lateral extent (e.g., a diameter) that is greater than the lateral extent of the first fluid passageway 1510, such that when the outer frame 1550 is disposed in the receptacle, an inner wall of the outer frame has a lateral extent that is similar to the lateral extent of the first fluid passageway 1510. In some implementations, the outer frame can be press fit into the receptacle. In some implementations the outer frame 1550 can be welded to the portion of the base plate 1502 that defines the receptacle. In some implementations, after the outer frame 1550 of the filter 1540C is placed in the receptacle, a foil 1542 can be placed over the outer frame 1550 and then attached (e.g., welded) to the base plate 1502.

In different implementations, the outer frame 1550 can be made of different materials. For example, if the outer frame 1550 is to be welded to a titanium base plate 1502, the outer frame 1550 can be made of titanium. In another example, if the outer frame 1550 is to be securely press fit into a receptacle, the outer frame 1550 can be made of a compliant material, for example, plastic, rubber, etc.

The material of the filter 1540C supported by the outer frame 1550, which includes a plurality of small openings or passages through fluid passes, can be made of different materials, which need not be identical or similar to the materials of the outer frame 1550. For example, the material can include metal (e.g., titanium, gold, etc.). In another example the material can include ceramic material. In another example, the material can include plastic.

In some implementations, the thickness of the material of the filter, which includes the plurality of small openings or passages through which fluid passes, in the direction of the fluid flow through the filter can be greater than three times the mean lateral extent of the openings or passages through which the fluid passes. Thus, the openings or passages of the materials can operate more as tubes through which the fluid passes than as apertures in a thin plane of material. In some implementations, walls of the openings or passages of the material can be textured or treated to promote the adhesion of particulate matter, while also permitting the fluid to pass through the openings or passages. For example, the walls of the openings or passages can have a surface texture or roughness that facilitates the adhesion of particulate matter, and the service of the openings or passages can include a hydrophobic coating to encourage the passage of fluid through the openings or passages.

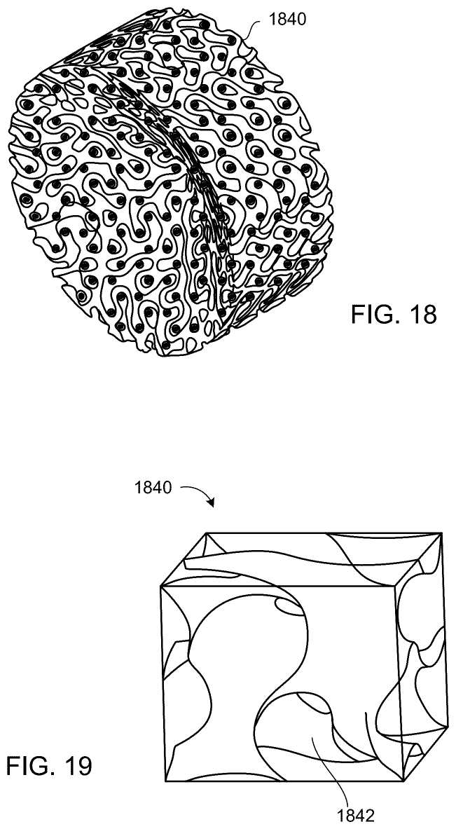

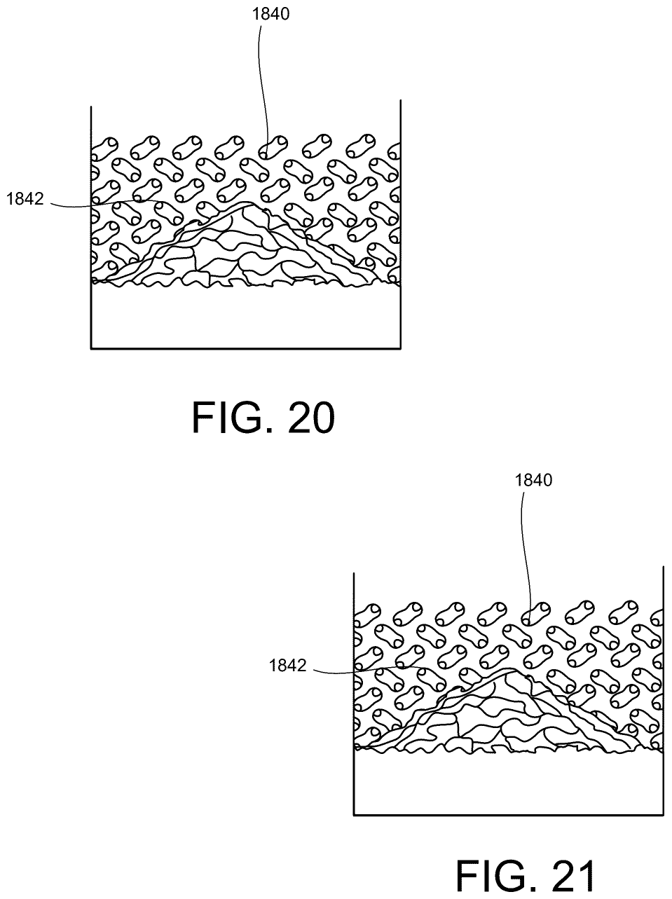

FIGS. 18-21 illustrate a material of a filter 1840 according to an example implementation, where the filter 1840 is a gyroid filter. In some implementations, the filters 1540C shown in FIGS. 17A-17C can include gyroid filters. As illustrated in FIG. 19, the filter 1840 includes or defines lumens 1842 that extend through the filter 1840 and are non-linear. The lumens 1842 can follow a tortuous path as they extend through the filter 1840, such that an average distance traveled fluid along a tortuous path through the gyroid filter can be substantially greater than a thickness of the filter. FIG. 20 is a front view of the filter 1840. FIG. 21 is a side view of the filter 1840. In some implementations, and average distance of fluid through the gyroid filter can be more than two times thickness of the gyroid filter in a direction parallel to an average flow direction of fluid through the gyroid filter. In some implementations, the gyroid filter can be created through a three-dimensional printing process, for example, by sintering material (e.g., ceramic material, metallic material, etc.) in a three-dimensional deposition process.

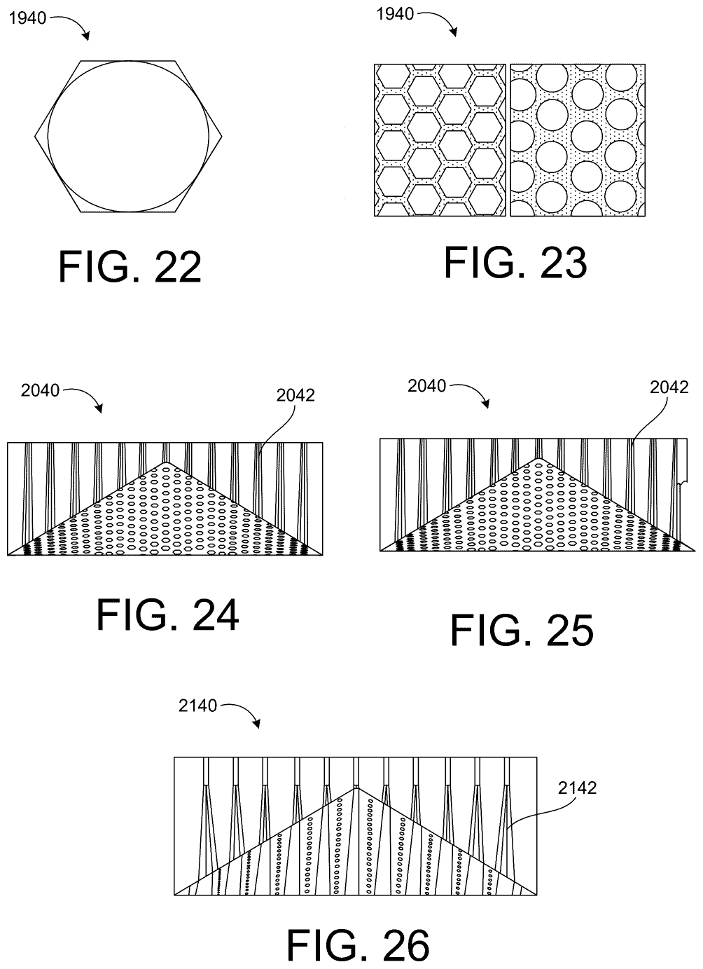

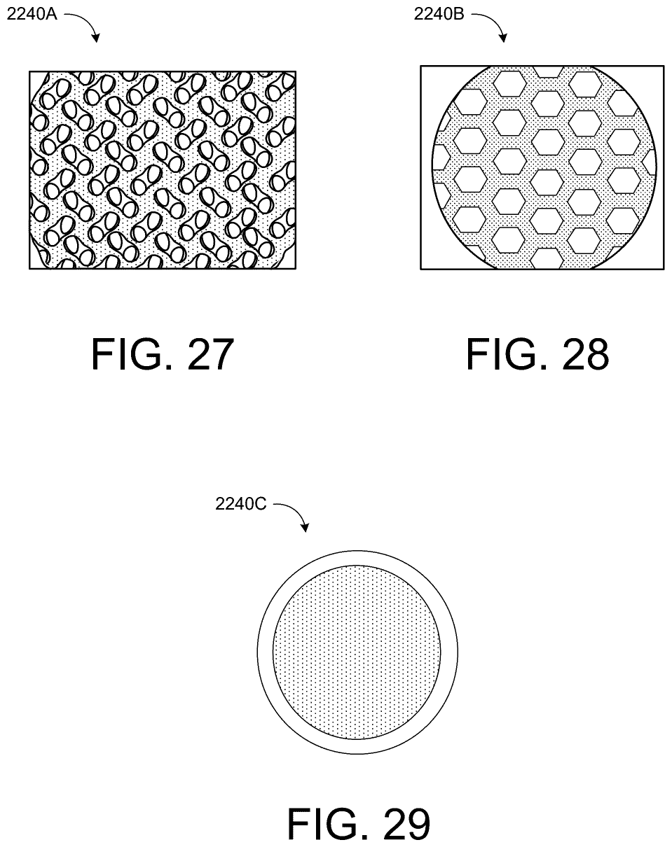

FIGS. 22-29 illustrate filters according to different embodiments. As illustrated in FIGS. 22-23, the filter 1940 may have different shaped openings. As illustrated in FIG. 24, the filter 2040 defines lumens 2042 that are linear. As illustrated in FIG. 25, the filter 2040 defines conical or tapered lumens 2042. As illustrated in FIG. 26, the filter 2140 defines Y-shaped lumens 2142. In some embodiments, the filters or flow modifiers are configured to modify or restrict the flow by blocking the flow, causing turbulence, or via another method. FIGS. 27-29 illustrate filters 2240A, 2240B, and 2240C that include openings or lumens of different shapes. In some embodiments, the filters may enhance the flow, for example by inducing laminar flow or via another method.

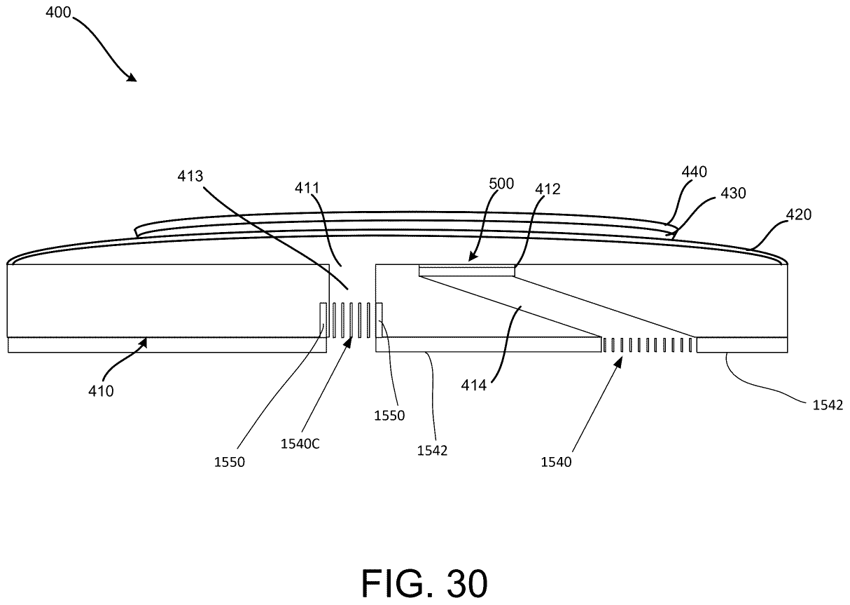

In addition to being used in the pumps described herein, the filters described herein also can be used in the valves described herein. For example, FIG. 30 is cross-sectional view of the valve device 400 shown in FIG. 5A, but also including a filter 1540 located at an end of the second fluid passageway 414 and a filter 1540C located within the first fluid passageway 413. The filters described herein also may be utilized in other valve structures described herein.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable fluid-operated device configured to control fluid flow between a fluid reservoir and an inflatable member, the device comprising:
a base plate;
a deformable diaphragm;
a fluid chamber defined between the base plate and the deformable diaphragm, the fluid chamber being in fluidic connection with the fluid reservoir and with the inflatable member, wherein the base plate defines a first fluid passageway configured for routing fluid from the fluid reservoir into the fluid chamber and a second fluid passageway configured for routing fluid from the fluid chamber to the inflatable member;
a fluid filter disposed within or at an end of the first fluid passageway or disposed within or at an end of the second fluid passageway, wherein the end of the first fluid passageway is distal from the fluid chamber and wherein the end of the second fluid passageway is distal from the fluid chamber; and
a piezoelectric element attached to the deformable diaphragm, the piezoelectric element being operable to repeatedly change a volume of the fluid chamber by deforming the deformable diaphragm to pump fluid from the fluid reservoir to the inflatable member.

2. The implantable fluid-operated device of claim 1, wherein the fluid filter includes a metal foil having a pattern of openings configured to permit fluid to flow through the openings and configured to block particulates having a characteristic size larger than a threshold size from flowing through the openings.

3. The implantable fluid-operated device of claim 2, wherein the metal foil includes titanium.

4. The implantable fluid-operated device of claim 3, wherein the base plate includes titanium and wherein the metal foil is welded to the base plate with openings of the pattern of openings being located at the end of the first fluid passageway or at the end of the second fluid passageway.

5. The implantable fluid-operated device of claim 2, wherein the pattern of openings is stamped into the metal foil.

6. The implantable fluid-operated device of claim 2, wherein the pattern of openings is laser-etched into the metal foil.

7. The implantable fluid-operated device of claim 2, wherein the pattern of openings is chemically etched into the metal foil.

8. The implantable fluid-operated device of claim 1, wherein the filter is a gyroid filter.

9. The implantable fluid-operated device of claim 8, wherein an average path distance of fluid through the gyroid filter is more than two times a thickness of the gyroid filter in a direction parallel to an average flow direction of fluid through the gyroid filter.

10. The implantable fluid-operated device of claim 8, wherein the gyroid filter includes ceramic material.

11. The implantable fluid-operated device of claim 8, wherein the gyroid filter is located within the first fluid passageway.

12. The implantable fluid-operated device of claim 8, wherein the gyroid filter is located within the second fluid passageway.

13. The implantable fluid-operated device of claim 8, wherein inner walls of the gyroid filter include a hydrophobic coating.

14. The implantable fluid-operated device of claim 1, wherein first fluid passageway is tapered, such that it has a larger cross-sectional area at the end of the first fluid passageway that is distal to the fluid chamber than an end of the first fluid passageway that is proximate to the fluid chamber.

15. An implantable fluid-operated device configured to control fluid flow between a fluid reservoir and an inflatable member, the device comprising:
a titanium base plate, wherein the base plate defines a first fluid passageway through the base plate and defines a second fluid passageway through the base plate;
a diaphragm coupled to the base plate;
a fluid chamber defined between the base plate and the diaphragm;
a first opening formed in the base plate, the first opening connecting the fluid chamber to a first fluid passageway;
a second opening formed in the base plate, the second opening connecting the fluid chamber to a second fluid passageway;
a fluid control device provided at one of the first opening or the second opening, wherein, in an open state of the fluid control device, the first fluid passageway, the fluid chamber, and the second fluid passageway are fluidically connected to guide fluid through the implantable fluid-operated device in a first fluid flow direction, and wherein, in a closed state of the fluid control device, the fluid control device blocks the flow of fluid the flow of fluid between the first fluid passageway and the second fluid passageway in response to a force applied in a second fluid flow direction, opposite the first fluid flow direction; and
a fluid filter disposed within or at an end of the first fluid passageway or disposed within or at an end of the second fluid passageway, wherein the end of the first fluid passageway is distal from the fluid chamber and wherein the end of the second fluid passageway is distal from the fluid chamber.

16. The implantable fluid-operated device of claim 15, wherein the fluid filter includes a titanium foil having a pattern of openings configured to permit fluid to flow through the openings and configured to block particulates having a characteristic size larger than a threshold size from flowing through the openings, wherein titanium foil is welded to the base plate with openings of the pattern of openings being located at the end of the first fluid passageway or at the end of the second fluid passageway.

17. The implantable fluid-operated device of claim 15, wherein the filter is a gyroid filter.

18. The implantable fluid-operated device of claim 17, wherein an average path distance of fluid through the gyroid filter is more than two times a thickness of the gyroid filter in a direction parallel to an average flow direction of fluid through the gyroid filter.

19. The implantable fluid-operated device of claim 17, wherein inner walls of the gyroid filter include a hydrophobic coating.

20. The implantable fluid-operated device of claim 15, wherein first fluid passageway is tapered, such that it has a larger cross-sectional area at the end of the first fluid passageway that is distal to the fluid chamber than an end of the first fluid passageway that is proximate to the fluid chamber.
